SEQUENCE LISTING (21 sequences)

SEQ ID NO: 1 - protein, 712 aa, Bos taurus
MSQWYELQQLESKYLEQVHQLYDDSFPMEIRQYLAQWLEKQDWEHAANDVSFATIRFHDLLSQLDDQYSRFSLENNFLLQHNIRKSKRNLQDNFQEDPIQMSMIICNCLKEERKILDHAQRISQAQSGNIQSTVMLDKQKELDSKVRNVKDKVMSIEHEIKTLEDLQDEYDFKCKTLQNREHETNGVAKNDQKQEQLLLQKMYLMLDNKRKEVVLKIIELLNATELTQKALINDELVEWKRRQQSACIGGPPNACLDQLQNWFTIVAESLQQVRQQLKKLEELEQKYTYEHDPITKNKQALWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGARTNEGPLIVTEELHSLSFETQLCQPGLVIDLETTSLPVVVISNVSQLPSGWASILWYNMLVAEPRNLSFFLNPPCARWSQLSEVLSWQFSSVTKRGLNVDQLNMLGEKLLGPNAGPDGLIPWTRFCKENINDKNFPFWLWIESILELIKKHLLALWNDGCIVGFISKERERALLKDQQPGTFLLRFSESCREGAITFTWVERSQNGGEPYFHAVEPYTKKELSAVTFPDIIRNYKVMAAENIPENPLKFLYPNIDKDHAFGKYYSRPKEAPEPMELDGPKGTGYIKTELISVSEV

SEQ ID NO: 2 - protein, 751 aa, Bos taurus
MSQWNQVQQLEIKFLEQVDQFYDDNFPMEIRHLLAQWIENQDWEAASNNETMATILLQNLLIQLDEQLGRVSKEKNLLLIHNLKRIRKVLQGKFHGNPMHVAVVISNCLREERRILAAANMPVQGPLEKSLQSSSVSERQRNVEHKVAAIKNSVQMTEQDTKYLEDLQDEFDYRYKTIQTMDQGDKNSALMNQEVLTLQEMLNSLDFKRKEALSKMTQIVNETDLLMNSMLVEELQDWKRRQQIACIGGPLHSGLDQLQNCFTLLAESLFQLRRQLEKLEEQSSKMTYEGDPIPLQRAHLLERVTFLIYNLFKNSFVVERQPCMPTHPQRPMVLKTLIQFTVKLRLLIKLPELNYQVKVKASIDKNASTLSNRRFVLCGTHVKAMSIEESSNGSLSVEFRHLQPKEMKSSAGNKGNEGCHMVTEELHSITFETQICLYGLTIDLETCSLPVVMISNVSQLPNAWASIIWYNVSTNDSQNLVFFNNPPSATLSQLLEVMSWQFSSYVGRGLNSDQLNMLAEKLTVQSSYNDGHLTWAKFCKEHLPGKSFTFWTWLEAILDLIKKHILPLWIDGYIMGFVSKEKERLLLKDKMPGTFLLRFSESHLGGITFTWVDHSENGEVRFHSVEPYNKGRLSALPFADILRDYKVIMAENIPENPLKYLYPDIPKDKAFGKHYSSQPCEVSRPTEKGDKGYVPSVFIPISTISSSRSDSTEPHSPSDLLPMSPSVYAVLRENLSPTTIETAMKSPYSAE

SEQ ID NO: 3 - protein, 1156 aa, Bos taurus
MACPWQFLFKIKSQKVDLATELDINNNVGKFYQPPSSPVTQDDPKRHSPGKHGNESPQPLTGTVKTSPESLSKLDAPPSACPRHVRIKNWGSGVTFQDTLHQKAKGDLSCKSKSCLASIMNPKSLTIGPRDKPTPPDELLPQAIEFVNQYYGSFKEAKIEEHLARVEAVTKEIETTGTYQLTGDELIFATKQAWRNAPRCIGRIQWSNLQVFDARSCSTAQEMFEHICRHVRYATNNGNIRSAITVFPQRSDGKHDFRVWNAQLIRYAGYQMPDGSIRGDPANVEFTQLCIDLGWKPKYGRFDVLPLVLQADGRDPELFEIPPDLVLEVPMEHPRYEWFRELELKWYALPAVANMLLEVGGLEFPGCPFNGWYMGTEVGVRDFCDAQRYNILEEVGRRMGLETHKVASLWKDRAVVEINVAVLHSFQKQNVTIMDHHSAAESFMKYMQNEYRSRGGCPADWIWLVPPISGSITPVFHQEMLNYVLSPFYYYQVEPWKTHVWQDERRRPQRREIRFKVLVKAVFFASVLMHKAMASRVRATILFATETGRSETLAQDLGALFSCAFNPKVLCMDQYQLSHLEEEQLLLVVTSTFGNGDSPGNGEKLKKSLLMLKELTNTFRYAVFGLGSSMYPQFCAFAHDIDQKLSQLGASQLAPTGEGDELSGQEEAFRSWAVQTFKAACETFDVSGKHHIEIPKLYTSNVTWDPQHYRLVQDSEPLDLNKALSSMHAKHVFTMRLKSQQNLQSPKSSRTTLLVELSCEGSQAPSYLPGEHLGVFPCNQPALVQGILERVVDGPAPHQPVRLETLCENGSYWVKDKRLPPCSLSQALTYFLDITTPPTQLLLRKLAQLATEEAEKQRLETLCQPSDYNKWKFTNSPTFLEVLEEFPSLRVSASFLLSQLPILKPRYYSISSSRDLTPTEIHLTVAVLTYRTRDGQGPLHHGVCSTWLSSLKPQDPVPCFVRSASGFQLPEDRSRPCILIGPGTGIAPFRSFWQQRLHEAEHKGLQGGRMTLVFGCRRPEEDHLYWEEMLEMARKGVLHEVHTAYSRLPDQPKVYVQDILRQRLAGEVLRVLHEEQGHLYVCGDVRMARDVARTLKQLMATALSLNEEQVEDYFFQLKNQKRYHEDIFGAVFPYEVKKDGAAGLPSNPRAPGAHRS

SEQ ID NO: 4 - protein, 322 aa, Bos taurus
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKSQRKERKSKSSRDARSKAKKKPYGEYSPDTFSDGLSSSTLPDDHSNYTVRSYMGQDLDIERTLTPALSPCGVSSTLPNWSIPVEIVPDSTSDLYNFQVSPMPSTSEAATDEDEEGKLTEDIMKLLEQTGWQQTSVDGKGYLLNEPGAQPTSVYGEFSCKEEPEVDSPGGYIGLISSDMKNMDPSWLDSLLTPVRLPSIQAIPCAP

SEQ ID NO: 5 - protein, 451 aa, Bos taurus
MNLEGGSRGGEFGMSSVSCGNGKLRQWLIDQIDSGKYPGLVWENEEKSIFRIPWKHAGKQDYNREEDAALFKAWALFKGKFREGIDKPDPPTWKTRLRCALNKSNDFEELVERSQLDISDPYKVYRIVPEGAKKGAKQLTLEDPQMPMSHPYSMPTPYPSLPAQQVHNYMIPPHDRGWREFVPDQPHAEIPYQCPVTFGPRGHHWQGPACENGCQVTGTFYACAPPESQAPGIPIEPSIRSAEALALSDCRLHICLYYREVLVKELTTSSPEGCRISHGHTYDASSLDQVLFPYPEDSSQRKNIEKLLSHLERGVVLWMAPDGLYAKRLCQSRIYWDGPLAICSDRPNKLERDQTCKLFDTQQFLSELQAFAHHGRPLPRFQVTLCFGEEFPDPQRQRKLITAHVEPLLARQLYYFAQQNSGHFLRGYDLPEHVGGPEDFHRPPRHSSIQE

SEQ ID NO: 6 - protein, 823 aa, Bos taurus
MEGAGGANDKKKISSERRKEKSRDAARSRRSKESEVFYELAHQLPLPHNVSSHLDKASVMRLTISYLRVRKLLDAGDLDIEDEMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQSQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWIETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSSNEKLQNINLAMSPLPASETPKPLRSSADPALNQEVALKLEPNPESLELSFTMPQIQDQPASPSDGSTRQSSPEPNSPSEYCFDVDSDMVNEFKLELVEKLFAEDTEAKNPFSTQDTDLDLEMLAPYIPMDDDFQLRSFDQLSPLENSSTSPQSASTNTVFQPTQMQEPPIATVTTTATSDELKTVTKDGMEDIKILIAFPSPPHVPKEPPCATTSPYSDTGSRTASPNRAGKGVIEQTEKSHPRSPNVLSVALSQRTTAPEEELNPKILALQNAQRKRKIEHDGSLFQAVGIGTLLQQPDDRATTTSLSWKRVKGCKSSEQNGMEQKTIILIPSDLACRLLGQSMDESGLPQLTSYDCEVNAPIQGSRNLLQGEELLRALDQVN

SEQ ID NO: 7 - protein, 480 aa, Bos taurus
MAEAPDRGTPRVLFGDWLLGEVSSGRYEGLRWLDAARTRFRVPWKHFARKDLGEADSRIFKAWAVARGRWPLRSGGGAPPIPESALRASWKTNFRCALRSTQRFVMLEDNSGDPTDPHKVYKISSEPGCPEGLGFDQGEDEALEDAPPARGGLLGPCLASDTGESLGHRLNPEPCPPSLAGDARDLLIQALQQSCLEDHLLDLTPPEAPDAGPPPEPWQPLEAEPHMGASASACTPMAGEPPLAGPGYSQLGLQPEPSLGALDLSILYKGRTVLQEVVGRPRCVPLYGPSAVAGGAPAPQQVAFPSPAGLPDQKQLHYTEKLLQHVAPGLQLELRGPWLWARRLGKCKVYWEVGGPLGSASTSSPARLLPRDCDTPIFDFGTFFQELLEFRAQRRRGSPHYTIYLGFGQDLSVGRPKEKSLVLVKLEPWLCRAYLEAVQREGVSSLDSGSLSLCLSSSNSLYEDLEHFLEHFLMEVEQAA

SEQ ID NO: 8 - protein, 270 aa, Bos taurus
MLQACKMEGFPLVPPQPSEDLVPYDTDLYQRQTHEYYPYLSSDGESHSDHYWDFHPHHVHSEFESFPENHFTELQSVQPPQLQQLYRHMELEQMHVLEPPMAPPHANLSHQVYLPRMCLPYPSLSPARPSSDEEEGERQSPPLEVSDGEADGLEPGPGLLHGETGSKKKIRLYQFLLDLLRSGDMKDSIWWVDKDKGTFQFSSKHKEALAHRWGIQKGNRKKMTYQKMARALRNYGKTGEVKKVKKKLTYQFSGEVLGRGGLAERRHPPH

SEQ ID NO: 9 - protein, 672 aa, Bos taurus
MAEAPASPAPISPLEVELDPEFEPQSRPRSCTWPLQRPELQGSPAKPSGEAAADSMIPEEEDDEDDEDGGGRAGSAMAIGGGGGGPLGSGLLLEDSARLLAPGGQDPGSGPAPAAGALSGGTQTPLQPQQPLPPPQPGTAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPILASTELDDVQDDDAPLSPMLYSSSASLSPSVSKPCTVELPRLTDMAGTMNLNDGLADNLMDDLLDNIALPASQPSPPGGLMQRSSSFPYTTKGSGLGSPTSSFSSAVFGPSSLNSLRQSPMQTIQENKPATFSSMSHYGNQTLQDLLTSDSLSHSDVMMTQSDPLMSQASTAVSAQNSRRNVMLRSDPMMSFAAQPNQGSLVNQNLLHHQHQTQGALGGSRALSNSVSNMGLSDSSSLGSAKHQQQSPVSQSMQTLSDSVSGSSLYSTSANLPVMGHEKFPSDLDLDMFNGSLECDMESIIRSELMDADGLDFNFDSLISTQNVVGLNVGSFTGAKQASSQSWVPG

SEQ ID NO: 10 - protein, 591 aa, Bos taurus
MASGGFNPCIEIIEQPRQRGMRFRYKCEGRSAGSIPGEHSTDNNRTYPSIQILNYYGKGKVRITLVTKNDPYKPHPHDLVGKDCRDGYYEAEFGQERRPLFFQNLGIRCVKKKEVKDAVISRVRAGINPFNVPEQQLLDIEDCDLNVVRLCFQVFLPDEHGNLTTALPPVVSNPIYDNRAPNTAELRICRVNKNCGSVKGGDEIFLLCDKVQKDDIEVRFVLNDWEAKGVFSQADVHRQVAIVFKTPPYCKAIIEPVTVKMQLRRPSDQEVSESMDFRYLPDEKDTYGNKAKKQKTTLLFHKLWQDCGVNFPERPRPSPLGPTGEGRFIKKEPNLFSHGAVLPETSRPVSSQAESYYSSSASISSTLSHPASAMLPMGTQSSSGWSSVAHPTSRSVNTNSLSSFSTGTLSSNSQVIPPFLEMSDLNVSNACIYNNTNDIGRMEASSVSPADLYSISDASMLPNCPVNMITPSNDSMRETDNPRLVSMNLENPSCNSVLDPRDLRQLHQMSPSSMSTVTSSSTTAYVAQSEAFEGSDFNCADNSMINEAGPSNSTNANSHGFGPNSQYSGIGAMQNEQLSDSFAFEFFKVNL

SEQ ID NO: 11 - protein, 1545 aa, Bos taurus
  misc_feature (374)..(374): Xaa can be any naturally occurring amino acid
MPSDFISLLSADLDLESPKSLYSRDSLKLHPSQNFHRAGLLEESVYDLLPKELQLPPSRETPVASMSQTSGGEAGSPPPAVVAADASSAPSSSSMGGACSSFTTSSSPTIYSTSVTDSKAMQVESCSSALGVSNRGVSEKQLTSNTVQQHPSTPKRHTVLYISPPPEDLLDNSRMSCQDEGCGLESEQSCSMWMEDSPSNFSNMSTSSYNDNTEVPRKSRKRNPKQRPGVKRRDCEESNMDIFDADSAKAPHYVLSQLTTDNKGSSKAGNGTLENQKGTGVKKSPMLCGQYPVKSEGKELKIVVQPETQHRARYLTEGSRGSVKDRTQQGFPTVKLEGHNEPVVLQVFVGNDSGRVKPHGFYQACRVTGRNTTXCKEVDIEGTTVIEVGLDPSNNMTLAVDCVGILKLRNADVEARIGIAGSKKKSTRARLVFRVNITRKDGSTLTLQTPSSPILCTQPAGVPEILKKSLHSCSVKGEEEVFLIGKNFLKGTKVIFQENVSDENSWKSEAEIDMELFHQNHLIVKVPPYHDQHITLPVAVGIYVVTNAGRSHDVQPFTYTPDPAAVALNVNVKKEISSPARPCSFEEAMKAMKTTGCNLDKVNMLPNALITPLISSTMIKSEDITPMEVTAEKRSPSIFKTTKTVGSTQQTLENLSHIAGNGSFSSSSSHLTSENEKQQQIQPKAYNPETLTTIQTQDISQPGTFPAVSASSQLPSNDALLQQATQFQTRETQSREVLQSDGTVVNLSHLTETSQQQQQSPLQEQAQTLQQQISSNIFPSPNSVSQQLQNTIQHLQAGSFTGSTASGSNGNVDLVQQVLEAQQQLSSVLFSAPDGNENVQEQLSADIFQQVSQIQNSVSPGMFSSTEPAVHTRPDNLIAGRAESVHPQNENTLSNQQQQQQQQQVMDSSAAMVMEMQQSICQAAAQIQSELFPSSASANGNLQQSPVYQQTSHMMSALSANEDMQMQCELFSSPPAVSGNETTTTTTQQVATSGTTLFQTSNSGDGEETGAQAKQIQNSVFQTMVQMQHSGDSQPQVGLFSSTKSMISVQNSGTQQQGNGLFQQGNEMMSLQSGNFLQQSSHSQAQLFHPQNPIADPQNLSQETQGSIFHSPSPIVHSQTSTASSEQMQPPMFHSQNTMAVLQGSSVPQDQQSANIFLSQSPMNNLQTNTVAQEEQISFFAAQNSISPLQSTSNTEQQAAFQQQAPISHIQTPMLSQEQAQPSQQGLFQPQVSLGSLPPNPMPQNQQGTIFQSQHSIVAIQSNSPSQEQQQQQQQQQQQQSILFSNQNAMAPMASQKQPPPNMIFNPSQNPVANQEQQNQSIFHQQNNMAPMNQEQQPMQFQNQTTVSSLQNPGPAQSESSQTSLFHSSPQIQLVQGSPSSQEQQVTLFLSPASMSALQTSMNQQDMQQSPLYSPQNNMPGIQGATSSPQPQATLFHNTTGGTMNQLQNSPGSSQQTSGMFLFGIQNNCSQLLTSGPATLPDQLMAISPPGQPQNEGQPPVTTLLSQQMPENSPMASSINTNQNIEKIDLLVSLQNQGNNLTGSF

SEQ ID NO: 12 - protein, 348 aa, Bos taurus
MAEMSFLSSEVLGGDFVSPFDQLGLGAEESLGLLDDNLEVAKHFKHHGFSCDKAKAGSSEWLAVDWLVSDNSKEDAFSGTDWMVEKMDLKEFDFDILFSKDDLETMPDELLATLDDTCDLFQPLVQETNKEPPQIVNPIGHLPEGLPTIDQGAPFTFFQPLPPSPGTLSSTPDHSFSLELCSEVVIPEGDSKPDSTTTGFPQCIKEEDAPSDNDSGICMSPDSSLGSPQDSPSTSRGSPNKSLLSPGALSGSSRPKPYDPPGEKMVAAKVKGEKLDKKLKKMEQNKTAATRYRQKKRAEQEALTGECKELEKKNEALKEKADSLAKEIQYLKDQIEEVRKAREKKRVL

SEQ ID NO: 13 - protein, 680 aa, Bos taurus
MNFETSRCATLQYCPDPYIQRFVETPAHFSWKESYYRSTMSQSTQTSEFLSPEVFQHIWDFLEQPICSVQPIDLNFVDEPSENGATNKIEISMDCIRMQDSDLGDPMWPQYTNLGLLNSMDQQIQNGSSSTSPYNTDHAQNSVTAPSPYAQPSSTFDALSPSPAIPSNTDYPGPHSFDVSFQQSSTAKSATWTYSTELKKLYCQIAKTCPIQIKVMTPPPQGAVIRAMPVYKKAEHVTEVVKRCPNHELSREFNEGQIAPPSHLIRVEGNSHAQYVEDPITGRQSVLVPYEPPQVGTEFTTVLYNFMCNSSCVGGMNRRPILIIVTLETRDGQVLGRRCFEARICACPGRDRKADEDSIRKQQVSDSTKNGDGTKRPFRQNTHGIQMTSIKKRRSPDDELLYLPVRGRETYEMLLKIKESLELMQYLPQHTIETYRQQQQQQHQHLLQKQTSMQSQSSYGNSSPPLNKMNSMNKLPSVSQLINPQQRNALTPTTIPDGMGANIPMMGTHMPMAGDMNGLSPTQALPPPLSMPSTSHCTPPPPYPTDCSLVSFLARLGCSSCLDYFTTQGLTTIYQIEHYSMDDLASLKIPEQFRHAIWKGILDHRQLHDFSSPPHLLRTPSGASTVSVGSSETRGERVIDAVRFTLRQTISFPPRDEWNDFNFDMDARRNKQQRIKEEGE

SEQ ID NO: 14 - protein, 540 aa, Bos taurus
MAAAKAEMQLMSPLQISDPFGSFPHSPTMDNYPKLEEMMLSNGAPQFLGAAGAPEGSSGSSSGSSGGGGGGGGGSSSSNSNSSSAFNPQGEASEQPYEHLTAESFPDISLNNEKVLVETSYPSQTTRLPPITYTGRFSLEPAPNSGNTLWPEPLFSLVSGLVSMTNPPATSSSASSPAASSSASQSPPLSCAVQSNDSSPIYSAAPTFPTPNTDIFPEPQGQAFPGSAGPALQYPPPAYPGAKGGFQVPMIPDYLFPQQQGDLGLGTPDQKPFQGLESRTQQPSLTPLSTIKAFATQSGSQDLKALNSTYQSQLIKPSRMRKYPNRPSKTPPHERPYACPVESCDRRFSRSDELTRHIRIHTGQKPQCRISMRNFSRSDHLTTHIRTHTGEKPFACDICGRKFARSDERKRHTKIHLRQKDKKADKSAASAATSSLPSYPSPVATSYPSPATTSYPSPATTSYPSPVPTSYSSPGSSTYPSPVHNGFPSPSVATTYSSVPPAFPTQVSSFPSSAVTNSFSASTGLSDMTTTFSPRTIEIC

SEQ ID NO: 15 - protein, 153 aa, Bos taurus
MVRRFLITVRIRRANGPPRVRIFVVHIARAAGEWAAPSVRAAVALVLMASEEPAQSAAMHPRPGDDDGQRPRGRAAAAPRRGPQLRGPRHPHPTGARRRPGGLPGHAGGPAPSWSAAGCARCLGPPARGPGGGAGPPRRRPVPARGCRGHGRR

SEQ ID NO: 16 - protein, 1068 aa, Bos taurus
MDEDDPHAEGAAVVAAAGEALQALCQELNLDEGSAAEALDDFTAIRGNYSLEGEVIHWLACSLYVACRKSIIPTVGKGIMEGNCVSLTRILRSAKLSLIQFFSKMKKWMDMSNLPQEFRERIERLERNFEVSTVIFKKFEPIFLDIFQNPYEEPPKLPRSRKQRRIPCSVKELFNFCWTLFVYTKGNFRMIGDDLVNSYHLLLCCLDLIFANAIMCPNRQELLNPSFKGLPSNFQTADFRASEEPPCIIPVLCELHDGLLVEAKGIKEHYFKPYISKLFDRKILKGECLLDLCSFTDNSKAVNKEYEEYVLTVGDFDERIFLGADAEEEIGTPRKFTGDGPLGKLTAQANVECNLQHHFEKKTSFAPSTPLTGRRYLREKEAVITPVASATQSVSRLQSIVAGLKNAPSEQLINIFESCMRNPMENIMKIVKGIGETFCQHYTQSTDEQPGSHIDFAVNRLKLAEILYYKILETVMVQETRRLHGMDMSVLLEQDIFHHSLMACCLEIVLFAYSSPRTFPWIIEVLNLRPFYFYKVIEVVIRSEEGLSRDMVKHLNSIEEQILESLAWSHDSALWEALQASENRVPTCEEVIFPNNFETGSGGNVQGHLPMMPMSPLMHPRVKEVRTDSGSLRKDMQPLSPISVHERYSSPTAGSAKRRLFGEDPPKEILMDRIITEGTKLKIAPSSSITAENISISPGHSLLTMATAIVAGTTGHKVTIPLHGIANDAGEITLIPISMNTTQESKVESPVSLTAQSLIGASPKQTHLTKAQEVHPIGISKPKRTGSLALFYRKVYHLASVRLRDLCLKLDVSNELRRKIWTCFEFTLVHCPDLMKDRHLDQLLLCAFYIMAKVTKEERTFQEIMKSYRNQPQANSHVYRSVLLKSIPREVVAYSKNLNGDFEMTDCDLEDATKTPDCSSGPVKEERGDLIKFYNTIYVGRVKSFALKYDLSNQDHVMEAPPLSPFPHIKQQPGSPRRISQQHSIYVSPHKNGSGLTPRSALLYKFNGSPSKSLKDINNMIRQGEQRTKKRAITIDGDAESPAKRLCQENDDVLLKRLQDVVSERANH

SEQ ID NO: 17 - protein, 439 aa, Bos taurus
MPLNVSFANKNYDLDYDSVQPYFYCDEEENFYHQQQQSELQPPAPSEDIWKKFELLPTPPLSPSRRSGLCSPSYVAVASFSPRGDDDGGGGSFSSADQLEMVTELLGGDMVNQSFICDPDDETLIKNIIIQDCMWSGFSAAAKLVSEKLASYQAARKDGGSPSPARGHGGCSTSSLYLQDLSAAASECIDPSVVFPYPLNDSSSPKPCASPDSTAFSPSSDSLLSSAESSPRASPEPLALHEETPPTTSSDSEEEQEDEEEIDVVSVEKRQPPAKRSESGSPSAGSHSKPPHSPLVLKRCHVSTHQHNYAAPPSTRKDYPAAKRAKLDSGRVLKQISNNRKCASPRSSDTEENDKRRTHNVLERQRRNELKRSFFALRDQIPELENNEKAPKVVILKKATAYILSVQAEQQKLKSEIDVLQKRREQLKLKLEQIRNSCA

SEQ ID NO: 18 - protein, 555 aa, Bos taurus
MECLYCFLGFLLLAAGLPLDAAKRFHDVLSNERPSGYMREHNQLSGWSSDENDWNEKLYPVWKRGDSRWKSSWKGGRVQAVLTSDSPALVGSTITFAVNLVFPRCQKEDASGNIVYEKNCRNDTGASPDLYVYNWTAGTEDSDWGNDTSEGHHNVFPDGKPFPRPWKKNFVYVFHTLGQYFQKLGQCSVTISINTANVSLGPQIMEVTVYRRHRRAYVPIAKVKDVYVVTDQIPVFVTMSQKNNRNSSDETFLRDLPITFSVLIHDPSHFLNESAIYYKWNFGDNTGLFVSNNHTLNHTYVLNGTFSLNLTVQAEVPGPCPLPSPRPPKTTPPLVTAGDSTLELREIPDESCHITRYGYFKATITIVEGILEVNIIQVTDVPMPRPQPDNSLVDFVVTCHGSIPTEVCTIISDPSCQITQNPVCDPVAMELGDTCLLTVRRAFSGSGTYCMNLTLGNDASLALTSTLVSINSRDPASLLRTANGILVSLGCLAILVTVIAFLMYKKHKEYKPIENSPGIVIRGKGLNVFLNHAKTLFFPGNQEKDPLLKNQPGIL

SEQ ID NO: 19 - protein, 453 aa, Bos taurus
MAQWDDFPDQQEDTDSCTESVKFDARSVTALLPPHPKNGPTLQERMKSYKTALITLYLIVFVVLVPIIGIVAAQLLKWETKNCTVGSVNADISPSPEGKGNGSEDEMRFREAVMERMSNMESRIQYLSDNEANLLDAKNFQNFSITTDQRFNDVLFQLNSLLSSIQEHENIIGDISKSLVGLNTTVLDLQFSIETLNGRVQENAFKQQEEMRKLEERIYNASAEIKSLDEKQVYLEQEIKGEMKLLNNITNDLRLKDWEHSQTLKNITLLQGPPGPPGEKGDRGPPGQNGIPGFPGLIGTPGLKGDRGISGLPGVRGFPGPMGKTGKPGLNGQKGQKGEKGSGSMQRQSNTVRLVGGSGPHEGRVEIFHEGQWGTVCDDRWELRGGLVVCRSLGYKGVQSVHKRAYFGKGTGPIWLNEVFCFGKESSIEECRIRQWGVRACSHDEDAGVTCTT

SEQ ID NO: 20 - protein, 516 aa, Bos taurus
MEPAVSLAVCALLFLLWVRVKGLEFVLIHQRWVFVCLFLLPLSLIFDIYYYVRAWVVFKLSSAPRLHEQRVRDIQKQVREWKEQGSKTFMCTGRPGWLTVSLRVGKYKKTHKNIMINLMDILEVDTKKQIVRVEPLVTMGQVTALLTSIGWTLPVLPELDDLTVGGLIMGTGIESSSHRYGLFQHICTAYELVLADGSFVRCTPMENSDLFYAVPWSCGTLGFLVAAEIRIIPAKKYIKLRFEPVRGLEAICDKFTHESQQPENHFVEGLLYSLHEAVIMTGVMTDEAEPSKLNSIGNYYKPWFFKHVENYLKTNREGLEYIPLRHYYHRHTRSIFWELQDIIPFGNNPIFRYLFGWMVPPKISLLKLTQGETLRKLYEQHHVVQDMLVPMKCLPQALHTFHNDIHVYPIWLCPFILPSQPGLVHPKGDEAELYVDIGAYGEPRVKHFEARSCMRQLEKFVRSVHGFQMLYADCYMDREEFWEMFDGSLYHRLRKQLGCQDAFPEVYDKICKAARH

SEQ ID NO: 21 - protein, 433 aa, Bos taurus
MIWEFTVLLSLVLGTGAVPLEDPEDGGKHWVVIVAGSNGWYNYRHQADACHAYQIVHRNGIPDEQIIVMMYDDIANSEDNPTPGIVINRPNGSDVYQGVLKDYTGEDVTPKNFLAVLRGDAEAVKGVGSGKVLKSGPRDHVFVYFTDHGATGILVFPNEDLHVKDLNETIRYMYEHKMYQKMVFYIEACESGSMMNHLPPDINVYATTAANPRESSYACYYDEQRSTFLGDWYSVNWMEDSDVEDLTKETLHKQYQLVKSHTNTSHVMQYGNKSISAMKLMQFQGLKHQASSPISLPAVSRLDLTPSPEVPLSIMKRKLMSTNDLQESRRLVQKIDRHLEARNIIEKSVRKIVTLVSGSAAEVDRLLSQRAPLTEHACYQTAVSHFRSHCFNWHNPTYEYALRHLYVLVNLCENPYPIDRIKLSMNKVCHGYY

(19) United States
(12) Patent Application Publication
(10) Pub. No.: US 2022/0251511 A1
(43) Pub. Date: Aug. 11, 2022

(54) BOVINE MONOCYTE-DERIVED MACROPHAGE IN CULTURE SYSTEM AND METHODS FOR MEASURING INNATE IMMUNITY

(71) Applicant: University of Guelph, Guelph (CA)

(72) Inventors: Seyedmehdi Emam, Mont-Saint-Hilaire (CA); Bonnie Mallard, Ariss (CA)

(21) Appl. No.: 17/629,688

(22) PCT Filed: Jul. 17, 2020
(86) PCT No.: PCT/CA2020/050997

§ 371 Date: Jan. 24, 2022

Related U.S. Application Data
(60) Provisional application No. 62/878,377, filed on Jul. 25, 2019.
(60) Provisional application No. 62/941,927, filed on Nov. 29, 2019.

(51) Int. Cl. C12N 5/0786; C07K 14/535; A01K 67/02; C12Q 1/02; C12Q 1/6883; G01N 33/50

(52) U.S. Cl. CPC C12N 5/0645; C07K 14/535; A01K 67/02; C12Q 1/02; C12Q 1/6883; G01N 33/5055; C12N 2500/99; G01N 2800/7009; C12Q 2600/156; C12Q 2600/158; C12N 2506/115; C12N 2501/22; G01N 2800/24

(57) ABSTRACT

The present disclosure provides an in vitro method of generating bovine monocyte-derived macrophages from monocytes that produce nitric oxide and use as an indicator of innate immune response potential. The culture system includes culturing bovine monocytes in serum-free media supplemented with granulocyte-macrophage stimulating factor (GM-CSF) to generate monocyte-derived macrophages that produce nitric oxide.

### RELATED APPLICATIONS

This application claims the benefit of priority to U.S. Provisional Application Ser. No. 62/878,377 filed Jul. 25, 2019; and U.S. Provisional Application Ser. No.

62/941,927 filed Nov. 29, 2019, the contents of which is incorporated herein by reference in its entirety.

### FIELD

The present disclosure relates to a culture system for bovine monocyte-derived macrophages and methods thereof for measuring innate immune response potential.

### BACKGROUND

Genetic regulation of immune responses in mammals is exceptionally complex with about eighteen percent of the human genome (7,696 genes) annotated with the “immune response” term. In the bovine genome, this proportion increases to twenty-three percent with the total number of genes as 5,586 (ENSEMBL, release 93) (Zerbino et al., 2018). Analyzing host resistance or resilience in natural or experimental challenge models have been widely used to understand the genetic regulation of disease resistance (Min-Oo et al., 2007; Longley et al., 2011; Bishop, 2012; McManus et al., 2014; Abel et al., 2018). However, often the dynamic interaction between the host and pathogen, as well as the environmental effects which have a substantial effect on the outcome of infection, have resulted in various findings which shows the need for a well-defined phenotype for disease resistance (Minozzi et al., 2012; Greives et al., 2017). An alternative approach is to reduce the complexity of this system by removing the effects of the pathogen by measuring host immunocompetence without pathogen exposure (Thompson-Crispi et al., 2012b; Greives et al., 2017). This approach has revealed a substantial contribution of host genetics in the variation of bovine immune responses. The heritabilities of immunocompetence traits in cattle have been estimated to be approximately three to four folds larger than the heritability of disease occurrence and immune responses against specific pathogens (Clapperton et al., 2009; Ring et al., 2018). Evaluating key cellular components of the immune system in-vitro is another way to reduce the complexity of immunocompetence testing. Recently, immunophenotyping based on the performance of the cells of immune system in in-vitro models has been successfully carried out in humans. These cellular immunogenetics studies have identified phenotypic variation in cellular responses which then were linked to genetic variations and cellular mechanisms that control the resistance to bacterial infection in human populations (Ko et al., 2013; Alvarez et al., 2017; Wang et al., 2018). In cattle, immunophenotyping based on in-vivo responses of the adaptive immune system is currently available. Studies have shown that the phenotypes of adaptive immune responses are linked to the naturally occurring genetic variation and associated with resistance to infectious diseases (Thompson-Crispi et al., 2012a, 2014; Mallard et al., 2015). However, the innate arm of the immune system, has remained less investigated and there is still a need to identify a robust model to further the understanding of genetic regulation of the innate responses in cattle.

### SUMMARY

Health traits are complex, difficult to measure and very slow to improve due to low heritabilities. Measuring the performance of critical components of the immune system is an alternative approach to reduce the complexity of the health traits. The present inventors demonstrated the ability to assess in-vitro performance of monocyte-derived macrophages (MDMs) by measuring nitric oxide production (as an indicator of the respiratory burst function of macrophages) following exposure to two common bacterial pathogens of dairy cattle. The results showed that this cellular performance trait is highly heritable (h2: 0.776) with considerable variation among the individuals (CV: 70%) and may be a means to evaluate the genetic performance of bovine MDMs.

Accordingly, the present disclosure provides an in vitro method of generating bovine monocyte-derived macrophages from monocytes comprising a) culturing bovine monocytes in serum-free media supplemented with granulocyte-macrophage stimulating factor (GM-CSF) to generate bovine monocyte-derived macrophages (MDMs); and harvesting the bovine MDMs. In an embodiment, the GM-CSF is from 1-10 ng/mL, optionally about 5 ng/mL.

In an embodiment, the serum-free media further comprises the components 2-17 set out in Table 1. In one embodiment, the serum-free media further comprises 0.01-1 mM glycine, 0.01-1 mM L-alanine, 0.01-1 mM L-asparagine, 0.01-1 mM L-aspartic acid, 0.01-1 mM L-glutamic acid, 0.01-1 mM L-proline, 0.01-1 mM L-serine, 0.01-1 mM sodium pyruvate, 0.1-10 mg/L choline chloride, 0.1-10 mg/L D-calcium pantothenate, 0.1-10 mg/L folic acid, 0.1-10 mg/L nicotinamide, 0.1-10 mg/L pyridoxal hydrochloride, 0.01-1 mg/L riboflavin, 0.1-10 mg/L thiamine hydrochloride and 0.2-20 mg/L i-inositol. In a particular embodiment, the serum-free media further comprises 0.1 mM glycine, 0.1 mM L-alanine, 0.1 mM L-asparagine, 0.1 mM L-aspartic acid, 0.1 mM L-glutamic acid, 0.1 mM L-proline, 0.1 mM L-serine, 0.1 mM sodium pyruvate, 1 mg/L choline chloride, 1 mg/L D-calcium pantothenate, 1 mg/L folic acid, 1 mg/L nicotinamide, 1 mg/L pyridoxal hydrochloride, 0.1 mg/L riboflavin, 1 mg/L thiamine hydrochloride and 2 mg/L i-inositol.

In one embodiment, the cells in a) are cultured for 4-8 days, optionally about 6 days.

In another embodiment, the method further comprises obtaining a blood sample from the bovine animal and purifying the bovine monocytes prior to a). In an embodiment, the bovine animal is a cow. In another embodiment, the bovine animal is a bull.

Also provided herein is a method of measuring innate immune response potential of a bovine animal comprising a) generating bovine MDMs by the method disclosed herein; b) exposing the bovine MDMs to a bacterial pathogen to induce respiratory burst; c) collecting supernatant of the culture; and d) measuring molecules that are produced by respiratory burst, such as nitric oxide or reactive oxygen species in the supernatant as a measure of innate immune response potential. In an embodiment, d) measures nitric oxide production in the supernatant as a measure of innate immune response potential.

In one embodiment, the bovine MDMs are exposed to the bacterial pathogen for 18 to 72 hours, optionally 48 hours.

In one embodiment, the bacterial pathogen is live attenuated bacteria. In another embodiment, the bacterial pathogen is an inactivated bacteria. In an embodiment, the bacteria is Gram-negative bacteria such as E. coli, Klebsiella spp, Serratia spp, Enterobacter spp. In another embodiment, the bacteria is Gram-positive bacteria such as Staphylococcus spp., or Streptococcus spp., optionally S. aureus.

In yet another embodiment, the bacterial pathogen is a purified microbial component such as lipopolysaccharide, peptidoglycan, flagellin, lipoteichoic acid, and zymosan, or a synthetic reagent that resembles bacterial components such as pam3csk4, poly(I:C), CRX-527, and Tri-DAP.

In another embodiment, the bacterial pathogen is inactivated E. coli and the bovine MDMs are exposed to the inactivated E. coli at a multiplicity of infection of 1-50, optionally 5. In yet another embodiment, the bacterial pathogen is inactivated S. aureus and the bovine MDMs are exposed to the inactivated S. aureus at a multiplicity of infection of 1-50, optionally 10.

Also provided herein is a method of measuring innate immune response potential of a bovine animal comprising a) generating bovine MDMs by the method disclosed herein; b) exposing the bovine MDMs to fluorescently-labelled bacteria; and c) measuring bacterial uptake of the MDMs as a measure of innate immune response potential.

In an embodiment, the bacteria is Gram-negative bacteria such as E. coli, Klebsiella spp, Serratia spp, Enterobacter spp. In another embodiment, the bacteria is Gram-positive bacteria such as Staphylococcus spp., or Streptococcus spp, optionally S. aureus. In one embodiment, the fluorescent label is pHRodo Green, pHRodo Red, mCherry, FITC, or Alexa Fluor.

Even further provided herein is a method of selecting bovine animals for breeding comprising measuring innate immune response potential of a bovine animal by a method disclosed herein; and selecting the bovine animal for breeding if the innate immune response is high. In an embodiment, high innate immune response potential is greater than 11 pM NO production as measured in the methods disclosed herein when 0.4±0.05e6 cells per 1 cm of culture area in 1 mL of culture medium are used. In another embodiment, high innate immune response potential is determined as an increase of respiratory burst, optionally indicated by NO production or reactive oxygen species, compared to a control known to have low innate immune response potential.

Yet further provided herein is a method of ranking bovine animals for innate immune response potential comprising measuring the innate immune response potential of a group of bovine animals by a method disclosed herein; and ranking the bovine animals in order of innate immune response potential.

Even further provided is a method of screening for a gene expression profile for detecting optimal innate immune response potential of a bovine animal comprising

a) measuring the gene expression of a blood sample from a bovine animal that has been identified as having high innate immune response potential by a method disclosed herein;

b) measuring the gene expression of a blood sample from a bovine animal that has been identified as having a low innate immune response potential by a method disclosed herein;

c) identifying genes that are differentially expressed between a) and b) to determine the gene expression profile for detecting optimal innate immune response potential of a bovine animal.

In an embodiment, high innate immune response potential is greater than 11 μM NO production and low innate immune response potential is less than 6 μM as measured in the methods disclosed herein when 0.4±0.05e6 cells per 1 cm of culture area in 1 mL of culture medium are used. In another embodiment, high innate immune response potential is determined as an increase of respiratory burst, optionally indicated by NO production or reactive oxygen species, compared to a control known to have low innate immune response potential.

Also provided is a method of screening for SNPs for detecting optimal innate immune response potential of a bovine animal comprising

a) determining a SNP profile of a tissue sample from a bovine animal that has been identified as having high innate immune response potential by a method disclosed herein;

b) determining a SNP profile of a tissue sample from a bovine animal that has been identified as having low innate immune response potential by a method disclosed herein;

c) identifying SNPs that are differentially found in a) and not b) to determine the SNPs for detecting optimal innate immune response of a bovine animal.

In an embodiment, high innate immune response potential is greater than 11 pM NO production and low innate immune response potential is less than 6 μM as measured in the methods disclosed herein when 0.4±0.05e6 cells per 1 cm of culture area in 1 mL of culture medium are used. In another embodiment, high innate immune response potential is determined as an increase of respiratory burst, optionally indicated by NO production or reactive oxygen species, compared to a control known to have low innate immune response potential.

Even further provided herein is a method of measuring innate immune response potential of a bovine animal comprising (a) generating bovine MDMs by the method disclosed herein; (b) exposing a test sample of the bovine MDMs to a bacterial pathogen for a period of time; (c) determining a test biomarker expression profile from the test sample, the test biomarker expression profile comprising the level of gene expression of at least one of STAT1, STAT4, iNOS, IRF1, IRF4 and HIF1A; (d) determining the level of similarity of the test biomarker expression profile to one or more control profiles, wherein a high level of similarity of the test biomarker expression profile to a high-innate control profile or a low level of similarity to a low-innate control profile indicates an increased likelihood of high innate immune response potential of the bovine animal; or a high level of similarity of the test biomarker expression profile to a low-innate control profile or a low level of similarity to a high innate control profile indicates an increased likelihood of low innate immune response potential of the bovine animal.

In an embodiment, the period of time for exposing the bovine MDMs to a bacterial pathogen in b) is between 1 to 4 hours. Optionally, the period of time for exposing the bovine MDMs to a bacterial pathogen is about 3 hours. In one embodiment, the test biomarker expression profile further comprises the gene expression level of at least one or more of IRF7, SPI1, FOXO3, REL, and NFAT5.

In another embodiment, the period of time for exposing the bovine MDMs to a bacterial pathogen in b) is between 12 to 24 hours. Optionally, the period of time for exposing the bovine MDMs to a bacterial pathogen is about 18 hours. In one embodiment, the test biomarker expression profile further comprises the gene expression level of at least one or more of ATF4, TP63, EGR1, CDKN2A, and RBL1. In another embodiment, the test biomarker expression profile further comprises the gene expression level of at least one or more of MYC, GPNMB, MSR1, DHCR24, and LGMN.

In an embodiment, the test biomarker expression profile is obtained by measuring the level of gene expression using Next-Generation Sequencing, Prob-Based Arrays (Microarray), Northern and Southern Blot Analysis and quantitative PCR (relative or absolute quantification).

Other features and advantages of the present disclosure will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Embodiments are described below in relation to the drawings in which:

FIG. 1 shows phenotypic characteristics of harvested cells after six days of in vitro incubation in serum-free media supplemented with recombinant bovine Granulocyte-macrophage colony-stimulating factor. The harvested cells were stained with Alexa Flour 647 conjugated anti-human CD-14 (Clone TÜK4), and phytoerythrin (PE) conjugated anti-bovine CD-205, separately. The cells were analyzed in BD Accuri™ C6 cytometer against one unstained harvested cells and one unstained blood mononuclear cells as the reference for autofluorescence in 533/30 filter excited by the blue laser. The data from the flow cytometer was analyzed and graphed using FlowJo (v. 10).

FIG. 2 shows phagocytic activity of monocyte-derived macrophages in response to in vitro treatment with E. coli and S. aureus. Monocyte-derived macrophages were harvested after 6 days and seeded in 96-well plates at concentration of 5×10⁴ per well. The cells were incubated overnight before treatment. Samples from each individual were treated with pH-rodo conjugated E. coli (MOI: 5) and S. aureus (MOI: 10), separately. The fluorescence intensity of each sample was corrected based on NucBlue stained control wells with no bacterial treatment. The graph represented log2 transformation of corrected fluorescence intensity as an indication of bacterial uptake. Only relative uptake of each bacterial strain can be compared among the samples in this graph due to the possible differences in saturation of the pH-rodo on the surface of each bacterial strain.

FIG. 3 shows nitric oxide production of monocyte-derived macrophages in response to in vitro treatment with E. coli and S. aureus. Monocyte-derived macrophages were harvested after 6 days in culture and seeded in 48-well plates at the concentration of 2×10⁵ per well. The cells were incubated overnight before bacterial treatment. Samples from each individual were treated with E. coli (MOI: 5) and S. aureus (MOI: 10), separately. The concentration of nitric oxide was determined using the Measure-iT™ High-Sensitivity Nitrite Assay Kit at 48 hours after treatment.

FIG. 4 shows distribution of the nitric oxide response. Nitrite concentration measured by the Measure-iT™ High-Sensitivity Nitrite Assay Kit in the supernatant of monocyte-derived macrophages 48 hours after treatment with Escherichia coli was transformed based on the method describe by K Krishnamoorthy et al. in 2008. The transformed data were used in subsequent statistical analysis.

FIG. 5a shows the interaction network among the transcription factors that were differentially regulated between the extreme phonotypes of high and low responder based on production of nitric oxide after 3 hours exposure to E. coli. The differentially regulated transcription factors were analyzed using the Search Tool for Retrieval of Interacting Genes/Proteins (STRING, v. 11). The inflammatory cluster and the hypoxia-related cluster is represented in their respective dashed-line boxes.

FIG. 5b shows the interaction network among the transcription factors that were differentially regulated between the extreme phonotypes of high and low responder based on production of nitric oxide after 18 hours exposure to E. coli. The differentially regulated transcription factors were analyzed using the Search Tool for Retrieval of Interacting Genes/Proteins (STRING, v. 11). The inflammatory cluster and the hypoxia-related cluster is represented in their respective dashed-line boxes.

### DETAILED DESCRIPTION

The present inventors provide a novel cell culture system for functional phenotyping of bovine Monocyte-Derived Macrophages (MDMs), cells which play a crucial role at all phases of inflammation, as well influence downstream immune responses. As indicators of MDMs function, phagocytosis and respiratory burst were tested in MDMs of 16 cows in response to two common bacterial pathogens of dairy cows, Escherichia coli (E. coli) and Staphylococcus aureus (S. aureus). Nitric oxide (NO⁻) production in MDMs was measured as an indicator of respiratory burst. Notable functional variations were observed among the individuals (coefficient of variation: 33% for phagocytosis and 70% in the production of NO⁻). The rank correlation analysis revealed significant, positive and strong correlation (rho=0.92) between NO⁻ production in response to E. coli and S. aureus, and positive but moderate correlation (rho=0.58) between phagocytosis of E. coli and S. aureus. To gain further insight into this trait, another 58 cows were evaluated solely for NO⁻response against E. coli. The pedigree of the tested animals was added to the statistical model and the heritability was estimated to be 0.776. Overall, the present inventors showed a strong effect of host genetics on the in-vitro activities of MDMs and the possibility of ranking Holstein cows based on the in-vitro functional variation of MDMs.

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. Further, “about”, as used herein, means a reasonable amount of deviation of the modified term such that the end result is not significantly changed. These terms of degree should be construed as including a deviation of at least +5% of the modified term if this deviation would not negate the meaning of the word it modifies or unless the context suggests otherwise to a person skilled in the art. When referring to a period such as a year or annually, it includes a range from 9 months to 15 months. All ranges disclosed herein are inclusive of the endpoints, and the endpoints are independently combinable with each other.

Methods

A serum-free model was developed to measure the functional performance of MDMs. The high level of respiratory burst, indicated by NO⁻ produced by MDMs in this culture system and high correlation that was observed between the two repeats of NO⁻ production in response to E. coli showed the robustness of this serum-free culture system.

Accordingly, the present disclosure provides an in vitro method of generating bovine monocyte-derived macrophages from monocytes comprising a) culturing bovine monocytes in serum-free media supplemented with granulocyte-macrophage stimulating factor (GM-CSF) to generate bovine monocyte-derived macrophages (MDMs); and b) harvesting the bovine MDMs.

As used herein the term “monocyte” refers to a type of blood mononuclear cell expressing CD14 marker on the surface.

As used herein the term “monocyte-derived macrophages” or “MDMs” refers to autofluorescent cells, expressing CD14 on their surface, able to produce detectable NO after bacterial or bacterial-derived component stimulation, able to engulf and internalized foreign particles via phagocytosis. A person skilled in the art would readily understand monocyte-derived macrophages have similar functional characteristics to mature macrophages. Thus, mature macrophages, such as those obtained from the bronchioles during lavage, may also be used to measure the respiratory burst.

In an embodiment, the GM-CSF is from 1-10 ng/mL, optionally about 5 ng/mL.

Any serum-free media useful for culturing and adhering monocytes may be used including, without limitation, AIM V Medium, CTSTM AIM V™ SFM, Macrophage-SFM, HuMEC Basal Serum-Free Medium, NutriStem® hPSC XF Medium, BIO-MPM-1 SFM, BIOTARGET™, DCCM-1 SFM, ImmunoCult™-SF Macrophage Medium.

In an embodiment, the serum-free media further comprises the components 2-17 set out in Table 1. In one embodiment, the serum-free media further comprises 0.01-1 mM glycine, 0.01-1 mM L-alanine, 0.01-1 mM L-asparagine, 0.01-1 mM L-aspartic acid, 0.01-1 mM L-glutamic acid, 0.01-1 mM L-proline, 0.01-1 mM L-serine, 0.01-1 mM sodium pyruvate, 0.1-10 mg/L choline chloride, 0.1-10 mg/L D-calcium pantothenate, 0.1-10 mg/L folic acid, 0.1-10 mg/L nicotinamide, 0.1-10 mg/L pyridoxal hydrochloride, 0.01-1 mg/L riboflavin, 0.1-10 mg/L thiamine hydrochloride and 0.2-20 mg/L i-inositol. In a particular embodiment, the serum-free media further comprises 0.1 mM glycine, 0.1 mM L-alanine, 0.1 mM L-asparagine, 0.1 mM L-aspartic acid, 0.1 mM L-glutamic acid, 0.1 mM L-proline, 0.1 mM L-serine, 0.1 mM sodium pyruvate, 1 mg/L choline chloride, 1 mg/L D-calcium pantothenate, 1 mg/L folic acid, 1 mg/L nicotinamide, 1 mg/L pyridoxal hydrochloride, 0.1 mg/L riboflavin, 1 mg/L thiamine hydrochloride and 2 mg/L i-inositol.

In one embodiment, the cells in a) are cultured for 4-8 days, optionally about 6 days prior to isolating the bovine MDMs. A person skilled in the art would readily understand the typical conditions for culturing the cells, for example, incubating at a temperature of 37° C. at 5% CO₂. A person skilled in the art would also understand that isolating the bovine MDMs comprises detaching and washing the cells and then resuspending the generated isolated bovine MDMs. A person skilled in the art would also understand that the culture medium has to be replaced every 2-3 days and all reagent should be pre-warmed to 37° C. before using them on the cultured cells. A person skilled in the art would understand that antibiotic must be supplied in the culture media and all procedure should be carried out aseptically.

In another embodiment, the method further comprises obtaining a blood sample from the bovine animal and purifying the bovine monocytes prior to a).

The term “bovine animal” as used herein refers to a cow or a bull. In one embodiment, the bovine animal is a cow. In another embodiment, the bovine animal is a bull.

Using the method of generating bovine MDMs disclosed herein, the present inventors showed a strong positive correlation in NO⁻ production (as an indicator of respiratory burst) (rho=0.92) along with the notable variation in NO⁻ response to E. coli (CV=70%) suggesting it may be suitable to use NO⁻ response to E. coli as a more general indicator of in-vitro bovine MDMs function and thus as an indicator of innate immune response potential.

Accordingly, provided herein is a method of measuring innate immune response potential of a bovine animal comprising a) generating bovine MDMs by the method disclosed herein; b) exposing the bovine MDMs to a bacterial pathogen to induce respiratory burst; c) collecting supernatant of the culture; and d) measuring molecules that are produced by respiratory burst, such as nitric oxide or a reactive oxygen species in the supernatant as a measure of innate immune response potential. In an embodiment, d) measures nitric oxide production in the supernatant as a measure of innate immune response potential.

The term “measuring respiratory burst” as used herein refers to measuring nitric oxide production or measuring reactive oxygen species as indicators of the respiratory burst.

The term “nitric oxide production” as used herein refers to the production of nitrogen monoxide (nitric oxide), nitrite, nitrate, and/or dinitrogen trioxide. Nitric oxide production may be measured by both direct or indirect methods. Nitric oxide production may be measured by direct methods, such as measurement of nitric oxide. Since nitric oxide is unstable, nitric oxide may alternatively be measured by indirect methods such as measuring any metabolites, derivatives, or downstream targets or products of nitric oxide which include, but are not limited to, nitrite, nitrate, or dinitrogen trioxide.

The term “measuring reactive oxygen species” as used herein refers to methods of measuring oxygen radicals, which include, but are not limited to, singlet oxygen, superoxide, hydrogen peroxide or hydroxyl radical.

The term “innate immune response” refers to a type of immune response that is nonspecific and typically arises immediately or within hours of an infection. The mechanisms of the innate immune response include, without limitation, physical barriers such as skin, chemicals in the blood, and cells that attack foreign cells, including macrophages, which are among the first responders to infections which can eliminate pathogens via phagocytosis and produce microbicidal components, such as nitric oxide (NO⁻).

In an embodiment, the bacterial pathogen is a live attenuated bacteria. In another embodiment, the bacterial pathogen is an inactivated bacteria. In an embodiment, the bacteria is Gram-negative bacteria such as E. coli, Klebsiella spp, Serratia spp, Enterobacter spp. In another embodiment, the bacteria is Gram-positive bacteria such as Staphylococcus spp., or Streptococcus spp., optionally S. aureus.

In yet another embodiment, the bacterial pathogen is a purified microbial component such as lipopolysaccharide, peptidoglycan, flagellin, lipoteichoic acid, zymosan, or a synthetic reagent that resembles bacterial components such as pam3csk4, poly(I:C), CRX-527, Tri-DAP.

The term “innate immune response potential” as used herein refers to the ability of the bovine animal to mount an innate immune response. The measure of respiratory burst, for example by NO production, and/or phagocytosis in the methods described herein are indicators of the performance of the innate immune system in the bovine animal. Differential expression of genes may provide another indicator of the innate immune system in the bovine animal. Differentially expressed genes may include, but are not limited to, transcription factors, which are highly conserved genes across many mammals. These transcription factors, which are highly conserved, are likely to provide an indicator of innate immune response potential in other mammals, including humans.

In one embodiment, the bovine MDMs are exposed to the inactivated bacteria for 18-72 hours, optionally 48 hours.

In another embodiment, the inactivated bacteria is inactivated E. coli and the bovine MDMs are exposed to the inactivated E. coli at a multiplicity of infection of 1-50, optionally 5. In yet another embodiment, the inactivated bacteria is inactivated S. aureus and the bovine MDMs are exposed to the inactivated S. aureus at a multiplicity of infection of 1-50, optionally 10.

The serum-free culture method for bovine MDMs developed herein was also effective in the evaluation of phagocytosis as an indicator of innate immune response potential.

Accordingly, also provided herein is a method of measuring innate immune response potential of a bovine animal comprising a) generating bovine MDMs by the method disclosed herein; b) exposing the bovine MDMs to fluorescently-labelled bacteria; and c) measuring bacterial uptake of the MDMs as a measure of innate immune response potential.

In an embodiment, the bacteria is Gram-negative bacteria such as E. coli, Klebsiella spp, Serratia spp, Enterobacter spp.

In another embodiment, the bacteria is Gram-positive bacteria such as Staphylococcus spp., or Streptococcus spp. In an embodiment, the Gram-positive bacteria is S. aureus.

The fluorescent label may be any label that allows detection of the phagocytosed bacteria. In one embodiment, the fluorescent label is pHRodo Green, pHRodo Red, FITC, mCherry, or Alexa Fluor.

Even further provided herein is a method of selecting bovine animals for breeding comprising measuring innate immune response potential of a bovine animal by a method disclosed herein; and selecting the bovine animal for breeding if the innate immune response potential is high. In an embodiment, high innate immune response potential is greater than 11 μM NO production as measured in the methods disclosed herein when 0.4±0.05e6 cells per 1 cm of culture area in 1 mL of culture medium are used. In another embodiment, high innate immune response potential is determined as an increase of respiratory burst, as measured by NO or reactive oxygen species, compared to a control.

The term “control” as used herein refers to a bovine animal that is known to have poor innate immune response potential. The control can also be a reference value or average reference value of bovine animal(s) that have poor innate immune response potential, typically less than 6 μM when 0.4±0.05e6 cells per 1 cm of culture area in 1 mL of culture medium are used. The control can also be referring to a technical replicate of MDM culture at concentration of 0.4±0.05e6 cells per 1 cm of culture area in 1 mL of culture medium that is not exposed to the stimulant and produces typically less than 1 μM of nitric oxide.

The term “control profile” as used herein refers to a biomarker expression profile of bovine MDMs that are generated from bovine which are known to have poor innate immune response potential or high innate immune response potential. The control can also be a reference value or average reference value of such bovine MDMs.

Yet further provided herein is a method of ranking bovine animals for innate immune response potential comprising measuring the innate immune response potential of a group of bovine animals by a method of disclosed herein; and ranking the bovine animals in order of innate immune response potential.

The high repeatability and heritability showed that the serum-free culture system disclosed herein and stimulation with GM-CSF is an effective method to evaluate bovine MDMs function in vitro and unmask the genetic effects.

Accordingly, provided is a method of screening for a gene expression profile for detecting optimal innate immune response potential of a bovine animal comprising

a) measuring the gene expression of a blood sample from a bovine animal that has been identified as having high innate immune response potential by a method disclosed herein;

b) measuring the gene expression of a blood sample from a bovine animal that has been identified as having a low innate immune response potential by a method disclosed herein;

c) identifying genes that are differentially expressed between a) and b) to determine the gene expression profile for detecting optimal innate immune response potential of a bovine animal.

In an embodiment, high innate immune response potential is greater than 11 μM NO production and low innate immune response potential is less than 6 μM as measured in the methods disclosed herein when 0.4±0.05e6 cells per 1 cm of culture area in 1 mL of culture medium are used. In another embodiment, high innate immune response potential is determined as an increase of respiratory burst, optionally indicated by NO production or reactive oxygen species, compared to a control known to have low innate immune response potential.

The term “gene expression profile” as used herein refers to the measurement of gene expression of a number of genes from a sample at one time, typically by measuring the mRNA expression levels in the sample. A “gene expression profile for detecting optimal innate immune response” refers to the pattern of gene expression that associates with an increased innate immune response compared to a control that has a poor innate immune response.

The terms “poor innate immune response” and “low innate immune response” are used interchangeably herein. A person skilled in the art would readily understand the terms to be equivalent and interchangeable.

The term “differentially expressed” refers to an increase or decrease in the measurable expression level of a gene as compared with the measurable expression level of the same gene in a second sample or control. In one embodiment, the differential expression can be compared using the ratio of the level of expression of the first sample as compared with the expression level of the second sample or control, wherein the ratio is not equal to 1.0. For example, a gene is differentially expressed if the ratio of the level of expression in a first sample as compared with a second sample is greater than or less than 1.0. For example, a ratio of greater than 1, 1.2, 1.5, 1.7, 2, 3, 5, 10, 15, 20 or more, ora ratio less than 1, 0.8, 0.6, 0.4, 0.2, 0.1, 0.05, 0.001 or less. In another embodiment the differential expression is measured using p-value. For instance, when using p-value, a gene is identified as being differentially expressed as between a first and second population when the p-value is less than 0.1, preferably less than 0.05, more preferably less than 0.01, even more preferably less than 0.005, the most preferably less than 0.001.

Also provided is a method of screening for SNPs for detecting optimal innate immune response potential of a bovine animal comprising

a) determining a SNP profile of a tissue sample from a bovine animal that has been identified as having high innate immune response potential by a method disclosed herein;

b) determining a SNP profile of a tissue sample from a bovine animal that has been identified as having low innate immune response potential by a method disclosed herein;

c) identifying SNPs that are differentially found in a) and not b) to determine the SNPs for detecting optimal innate immune response of a bovine animal.

The tissue sample may be any sample of tissue from the bovine animal that is able to provide the genetic information including, without limitation, a blood sample, a saliva sample, a hair sample.

In an embodiment, high innate immune response potential is greater than 11 μM NO production and low innate immune response potential is less than 6 μM as measured in the methods disclosed herein when 0.4±0.05e6 cells per 1 cm of culture area in 1 mL of culture medium are used. In another embodiment, high innate immune response potential is determined as an increase of respiratory burst, optionally indicated by NO production or reactive oxygen species, compared to a control known to have low innate immune response potential.

The term “SNP” or “single nucleotide polymorphism” as used herein refers to substitution of a single nucleotide at a specific position in the genome.

Even further provided herein is a method of measuring innate immune response potential of a bovine animal comprising:

a) generating bovine MDMs by the method disclosed herein;

b) exposing a test sample of the bovine MDMs to a bacterial pathogen for a period of time;

c) determining a test biomarker expression profile from the test sample, the test biomarker expression profile comprising the level of gene expression of at least one of STAT1, STAT4, IRF1, IRF4, iNOS and HIF1A; and d) determining the level of similarity of the test biomarker expression profile to one or more control profiles, wherein a high level of similarity of the test biomarker expression profile to a high-innate control profile or a low level of similarity to a low-innate control profile indicates an increased likelihood of high innate immune response potential of the bovine animal; or a high level of similarity of the test biomarker expression profile to a low-innate control profile or a low level of similarity to a high innate control profile indicates an increased likelihood of low innate immune response potential of the bovine animal.

In an embodiment, determining the test sample biomarker expression profile comprises measuring the expression level of the gene in the sample.

“Determining a test biomarker expression profile” can be readily accomplished by a person skilled in the art. In one embodiment, a probe that hybridizes to the mRNA sequence of the gene's nucleic acid sequence as can be used to detect and quantify the amount of the mRNA in the sample.

A nucleotide probe may be labelled with a detectable marker such as a radioactive label which provides for an adequate signal and has sufficient half life such as ³²P, ³H, ¹⁴C or the like. An appropriate label may be selected having regard to the rate of hybridization and binding of the probe to the nucleotide to be detected and the amount of nucleotide available for hybridization.

The term “hybridize” refers to the sequence specific non-covalent binding interaction with a complementary nucleic acid. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C. for 15 minutes, followed by a wash of 2.0× SSC at 50° C. for 15 minutes may be employed.

The stringency may be selected based on the conditions used in the wash step. For example, the salt concentration in the wash step can be selected from a high stringency of about 0.2× SSC at 50° C. for 15 minutes. In addition, the temperature in the wash step can be at high stringency conditions, at about 65° C. for 15 minutes.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.-16.6 (Log10 [Na+])+0.41(%(G+C)-600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% sequence identity, the final wash temperature will be reduced by about 5° C. Based on these considerations those skilled in the art will be able to readily select appropriate hybridization conditions. In an embodiment, stringent hybridization conditions are selected. By way of example the following conditions may be employed to achieve stringent hybridization: hybridization at 5x sodium chloride/sodium citrate (SSC)/5× Denhardt's solution/1.0% SDS at Tm-5° C. based on the above equation, followed by a wash of 0.2× SSC/0.1% SDS at 60° C. for 15 minutes. Moderately stringent hybridization conditions include a washing step in 3× SSC at 42° C. for 15 minutes. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 2000, Third Edition.

In another embodiment, primers that are able to amplify the gene sequence can be used, for example, in a quantitative PCR assay to determine the expression level of the gene.

As used herein, the term “amplify”, “amplifying” or “amplification” of DNA refers to the process of generating at least one copy of a DNA molecule or portion thereof. Methods of amplification of DNA are well known in the art, including but not limited to polymerase chain reaction (PCR), ligase chain reaction (LCR), self-sustained sequence replication (3SR), nucleic acid sequence based amplification (NASBA), strand displacement amplification (SDA), multiple displacement amplification (MDA) and rolling circle amplification (RCA).

The length and bases of primers for use in a PCR are selected so that they will hybridize to different strands of the desired sequence and at relative positions along the sequence such that an extension product synthesized from one primer when it is separated from its template can serve as a template for extension of the other primer into a nucleic acid of defined length. Primers which may be used in the disclosure are oligonucleotides, i.e., molecules containing two or more deoxyribonucleotides of the nucleic acid molecules of the disclosure which occur naturally as in a purified restriction endonuclease digest or are produced synthetically using techniques known in the art such as for example phosphotriester and phosphodiester methods (See Good et al. Nucl. Acid Res 4:2157, 1977) or automated techniques (See for example, Conolly, B. A. Nucleic Acids Res. 15:15(7): 3131, 1987). The primers are capable of acting as a point of initiation of synthesis when placed under conditions which permit the synthesis of a primer extension product which is complementary to a DNA sequence of the disclosure, i.e., in the presence of nucleotide substrates, an agent for polymerization such as DNA polymerase and at suitable temperature and pH. Preferably, the primers are sequences that do not form secondary structures by base pairing with other copies of the primer or sequences that form a hairpin configuration. The primer optionally comprises between about 7 and 25 nucleotides.

The primers may be labelled with detectable markers which allow for detection of the amplified products. Suitable detectable markers are radioactive markers such as P-32, S-35, 1-125, and H-3, luminescent markers such as chemiluminescent markers, preferably luminol, and fluorescent markers, preferably dansyl chloride, fluorcein-5-isothiocyanate, and 4-fluor-7-nitrobenz-2-axa-1,3 diazole, enzyme markers such as horseradish peroxidase, alkaline phosphatase, β-galactosidase, acetylcholinesterase, or biotin.

It will be appreciated that the primers may contain non-complementary sequences provided that a sufficient amount of the primer contains a sequence which is complementary to a nucleic acid molecule of the disclosure or oligonucleotide fragment thereof, which is to be amplified. Restriction site linkers may also be incorporated into the primers allowing for digestion of the amplified products with the appropriate restriction enzymes facilitating cloning and sequencing of the amplified product.

Methods of determining the similarity between biomarker expression profiles are well known in the art. Methods of determining similarity may in some embodiments provide a non-quantitative measure of similarity, for example, using visual clustering. In another embodiment, similarity may be determined using methods which provide a quantitative measure of similarity.

For example, in an embodiment, similarity may be measured using hierarchical clustering, optionally using Manhattan distance. For example, unsupervised hierarchical clustering of a sample with a high-innate control profile indicates similarity to the high-innate specific control profile. Likewise, unsupervised hierarchical clustering of a sample with a low-innate control profile indicates similarity to the low-innate specific control profile.

In another embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}}$$

In one embodiment, x and y are the gene expression values in a test biomarker expression profile and a control profile, respectively.

In an embodiment, a high level of similarity to the control profile is indicated by a correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

It will be appreciated that any “correlation value” which provides a quantitative scaling measure of similarity between biomarker expression profiles may be used to measure similarity.

As used herein the term “test sample” refers to a biological sample comprising the cultured monocyte derived macrophages generated from a bovine subject's tissue sample by the method disclosed herein. The tissue sample may be blood, bone marrow, or spleen. In an embodiment, the tissue sample is blood.

The term “STAT1” as used herein refers to Signal Transducer and Activator of Transcription 1. In one embodiment, STAT1 is of bovine origin. In another embodiment, STAT1 has the GenBank Accession NM_001077900 and the coding/amino acid sequence:

(SEQ ID NO: 1)
MSQWYELQQLESKYLEQVHQLYDDSFPMEIRQYLAQWLEKQDWEHAANDV
SFATIRFHDLLSQLDDQYSRFSLENNFLLQHNIRKSKRNLQDNFQEDPIQ
MSMIICNCLKEERKILDHAQRISQAQSGNIQSTVMLDKQKELDSKVRNVK
DKVMSIEHEIKTLEDLQDEYDFKCKTLQNREHETNGVAKNDQKQEQLLLQ
KMYLMLDNKRKEVVLKIIELLNATELTQKALINDELVEWKRRQQSACIGG
PPNACLDQLQNWFTIVAESLQQVRQQLKKLEELEQKYTYEHDPITKNKQA
LWDRTFSLFQQLIQSSFVVERQPCMPTHPQRPLVLKTGVQFTVKLRLLVK
LQELNYNLKVKVLFDKDVNERNTVKGFRKFNILGTHTKVMNMEESTNGSL
AAEFRHLQLKEQKNAGARTNEGPLIVTEELHSLSFETQLCQPGLVIDLET
TSLPVWISNVSQLPSGWASILWYNMLVAEPRNLSFFLNPPCARWSQLSEV
LSWQFSSVTKRGLNVDQLNMLGEKLLGPNAGPDGLIPWTRFCKENINDKN
FPFWLWIESILELIKKHLLALWNDGCIVGFISKERERALLKDQQPGTFLL
RFSESCREGAITFTWVERSQNGGEPYFHAVEPYTKKELSAVTFPDIIRN
YKVMAAENIPENPLKFLYPNIDKDHAFGKYYSRPKEAPEPMELDGPKGTG
YIKTELISVSEV

The term “STAT4” as used herein refers to Signal Transducer and Activator of Transcription 4. In one embodiment, STAT4 is of bovine origin. In another embodiment, STAT4 has the GenBank Accession NM_001083692 and the coding/amino acid sequence:

(SEQ ID NO: 2)
MSQWNQVQQLEIKFLEQVDQFYDDNFPMEIRHLLAQWIENQDWEAASNNE
TMATILLQNLLIQLDEQLGRVSKEKNLLLIHNLKRIRKVLQGKFHGNPMH
VAVVISNCLREERRILAAANMPVQGPLEKSLQSSSVSERQRNVEHKVAAI
KNSVQMTEQDTKYLEDLQDEFDYRYKTIQTMDQGDKNSALMNQEVLTLQE
MLNSLDFKRKEALSKMTQIVNETDLLMNSMLVEELQDWKRRQQIACIGGP
LHSGLDQLQNCFTLLAESLFQLRRQLEKLEEQSSKMTYEGDPIPLQRAHL
LERVTFLIYNLFKNSFVVERQPCMPTHPQRPMVLKTLIQFTVKLRLLIKL
PELNYQVKVKASIDKNASTLSNRRFVLCGTHVKAMSIEESSNGSLSVEFR
HLQPKEMKSSAGNKGNEGCHMVTEELHSITFETQICLYGLTIDLETCSLP
VVMISNVSQLPNAWASIIWYNVSTNDSQNLVFFNNPPSATLSQLLEVMSW
QFSSYVGRGLNSDQLNMLAEKLTVQSSYNDGHLTWAKFCKEHLPGKSFTF
WTWLEAILDLIKKHILPLWIDGYIMGFVSKEKERLLLKDKMPGTFLLRFS
ESHLGGITFTWVDHSENGEVRFHSVEPYNKGRLSALPFADILRDYKVIMA
ENIPENPLKYLYPDIPKDKAFGKHYSSQPCEVSRPTEKGDKGYVPSVFIP
ISTISSSRSDSTEPHSPSDLLPMSPSVYAVLRENLSPTTIETAMKSPYSA
E

The term “iNOS” as used herein refers to inducible nitric oxide synthase. In one embodiment, iNOS is of bovine origin. In another embodiment, iNOS has the GenBank Accession DQ676956 and the coding/amino acid sequence:

(SEQ ID NO: 3)
MACPWQFLFKIKSQKVDLATELDINNNVGKFYQPPSSPVTQDDPKRHSPGK
HGNESPQPLTGTVKTSPESLSKLDAPPSACPRHVRIKNWGSGVTFQDTLHQ
KAKGDLSCKSKSCLASIMNPKSLTIGPRDKPTPPDELLPQAIEFVNQYYGS
FKEAKIEEHLARVEAVTKEIETTGTYQLTGDELIFATKQAWRNAPRCIGRI
QWSNLQVFDARSCSTAQEMFEHICRHVRYATNNGNIRSAITVFPQRSDGKH
DFRVWNAQLIRYAGYQMPDGSIRGDPANVEFTQLCIDLGWKPKYGRFDVLP
LVLQADGRDPELFEIPPDLVLEVPMEHPRYEWFRELELKWYALPAVANMLL
EVGGLEFPGCPFNGWYMGTEVGVRDFCDAQRYNILEEVGRRMGLETHKVAS
LWKDRAVVEINVAVLHSFQKQNVTIMDHHSAAESFMKYMQNEYRSRGGCPA
DWIWLVPPISGSITPVFHQEMLNYVLSPFYYYQVEPWKTHVWQDERRRPQR
REIRFKVLVKAVFFASVLMHKAMASRVRATILFATETGRSETLAQDLGALF
SCAFNPKVLCMDQYQLSHLEEEQLLLVVTSTFGNGDSPGNGEKLKKSLLML
KELTNTFRYAVFGLGSSMYPQFCAFAHDIDQKLSQLGASQLAPTGEGDELS
GQEEAFRSWAVQTFKAACETFDVSGKHHIEIPKLYTSNVTWDPQHYRLVQD
SEPLDLNKALSSMHAKHVFTMRLKSQQNLQSPKSSRTTLLVELSCEGSQAP
SYLPGEHLGVFPCNQPALVQGILERVVDGPAPHQPVRLETLCENGSYWVKD
KRLPPCSLSQALTYFLDITTPPTQLLLRKLAQLATEEAEKQRLETLCQPSD
YNKWKFTNSPTFLEVLEEFPSLRVSASFLLSQLPILKPRYYSISSSRDLTP
TEIHLTVAVLTYRTRDGQGPLHHGVCSTWLSSLKPQDPVPCFVRSASGFQL
PEDRSRPCILIGPGTGIAPFRSFWQQRLHEAEHKGLQGGRMTLVFGCRRPE
EDHLYWEEMLEMARKGVLHEVHTAYSRLPDQPKVYVQDILRQRLAGEVLRV
LHEEQGHLYVCGDVRMARDVARTLKQLMATALSLNEEQVEDYFFQLKNQKR
YHEDIFGAVFPYEVKKDGAAGLPSNPRAPGAHRS

The term “IRF1” as used herein refers to interferon regulatory factor 1. In one embodiment, IRF1 is of bovine origin. In another embodiment, IRF1 has the GenBank Accession NM_001191261 and the coding/amino acid sequence:

(SEQ ID NO: 4)
MPITRMRMRPWLEMQINSNQIPGLIWINKEEMIFQIPWKHAAKHGWDINK
DACLFRSWAIHTGRYKAGEKEPDPKTWKANFRCAMNSLPDIEEVKDQSRN
KGSSAVRVYRMLPPLTKSQRKERKSKSSRDARSKAKKKPYGEYSPDTFSD
GLSSSTLPDDHSNYTVRSYMGQDLDIERTLTPALSPCGVSSTLPNWSIPV
EIVPDSTSDLYNFQVSPMPSTSEAATDEDEEGKLTEDIMKLLEQTGWQQT
SVDGKGYLLNEPGAQPTSVYGEFSCKEEPEVDSPGGYIGLISSDMKNMDP
SWLDSLLTPVRLPSIQAIPCAP

The term “IRF4” as used herein refers to interferon regulatory factor 4. In one embodiment, IRF4 is of bovine origin. In another embodiment, IRF4 has the GenBank Accession NM_001206162 and the coding/amino acid sequence:

(SEQ ID NO: 5)
MNLEGGSRGGEFGMSSVSCGNGKLRQWLIDQIDSGKYPGLVWENEEKSIF
RIPWKHAGKQDYNREEDAALFKAWALFKGKFREGIDKPDPPTWKTRLRCA
LNKSNDFEELVERSQLDISDPYKVYRIVPEGAKKGAKQLTLEDPQMPMSH
PYSMPTPYPSLPAQQVHNYMIPPHDRGWREFVPDQPHAEIPYQCPVTFGP
RGHHWQGPACENGCQVTGTFYACAPPESQAPGIPIEPSIRSAEALALSDC
RLHICLYYREVLVKELTTSSPEGCRISHGHTYDASSLDQVLFPYPEDSSQ
RKNIEKLLSHLERGVVLWMAPDGLYAKRLCQSRIYVVDGPLAICSDRPNK
LERDQTCKLFDTQQFLSELQAFAHHGRPLPRFQVTLCFGEEFPDPQRQRK
LITAHVEPLLARQLYYFAQQNSGHFLRGYDLPEHVGGPEDFHRPPRHSSI
QE

The term “HIF1A” as used herein refers to hypoxia inducible factor 1 subunit alpha. In one embodiment, HIF1A is of bovine origin. In another embodiment, HIF1A has the GenBank Accession NM_174339 and the coding/amino acid sequence:

(SEQ ID NO: 6)
MEGAGGANDKKKISSERRKEKSRDAARSRRSKESEVFYELAHQLPLPHNV
SSHLDKASVMRLTISYLRVRKLLDAGDLDIEDEMKAQMNCFYLKALDGFV
MVLTDDGDMIYISDNVNKYMGLTQFELTGHSVFDFTHPCDHEEMREMLTH
RNGLVKKGKEQNTQRSFFLRMKCTLTSRGRTMNIKSATWKVLHCTGHIHV
YDTNSNQSQCGYKKPPMTCLVLICEPIPHPSNIEIPLDSKTFLSRHSLDM
KFSYCDERITELMGYEPEELLGRSIYEYYHALDSDHLTKTHHDMFTKGQV
TTGQYRMLAKRGGYVWIETQATVIYNTKNSQPQCIVCVNYVVSGIIQHDL
IFSLQQTECVLKPVESSDMKMTQLFTKVESEDTSSLFDKLKKEPDALTLL
APAAGDTIISLDFGSNDTETDDQQLEEVPLYNDVMLPSSNEKLQNINLAM
SPLPASETPKPLRSSADPALNQEVALKLEPNPESLELSFTMPQIQDQPAS
PSDGSTRQSSPEPNSPSEYCFDVDSDMVNEFKLELVEKLFAEDTEAKNPF
STQDTDLDLEMLAPYIPMDDDFQLRSFDQLSPLENSSTSPQSASTNTVFQ
PTQMQEPPIATVTTTATSDELKTVTKDGMEDIKILIAFPSPPHVPKEPPC
ATTSPYSDTGSRTASPNRAGKGVIEQTEKSHPRSPNVLSVALSQRTTAPE
EELNPKILALQNAQRKRKIEHDGSLFQAVGIGTLLQQPDDRATTTSLSWK
RVKGCKSSEQNGMEQKTIILIPSDLACRLLGQSMDESGLPQLTSYDCEVN
APIQGSRNLLQGEELLRALDQVN

In an embodiment, the period of time for exposing the bovine MDMs to a bacterial pathogen in b) is between 1 to 4 hours, optionally about 3 hours. In one embodiment, the test biomarker expression profile further comprises the gene expression level of at least one or more of IRF7, SPI1, FOXO3, REL, and NFAT5.

The term “IRF7” as used herein refers to interferon regulatory factor 7. In one embodiment, IRF7 is of bovine origin. In another embodiment, IRF7 has the GenBank Accession NM_001105040 and the coding/amino acid sequence:

(SEQ ID NO: 7)
MAEAPDRGTPRVLFGDWLLGEVSSGRYEGLRWLDAARTRFRVPWKHFARK
DLGEADSRIFKAWAVARGRWPLRSGGGAPPIPESALRASWKTNFRCALRS
TQRFVMLEDNSGDPTDPHKVYKISSEPGCPEGLGFDQGEDEALEDAPPAR
GGLLGPCLASDTGESLGHRLNPEPCPPSLAGDARDLLIQALQQSCLEDHL
LDLTPPEAPDAGPPPEPWQPLEAEPHMGASASACTPMAGEPPLAGPGYSQ
LGLQPEPSLGALDLSILYKGRTVLQEVVGRPRCVPLYGPSAVAGGAPAPQ
QVAFPSPAGLPDQKQLHYTEKLLQHVAPGLQLELRGPWLWARRLGKCKVY
WEVGGPLGSASTSSPARLLPRDCDTPIFDFGTFFQELLEFRAQRRRGSPH
YTIYLGFGQDLSVGRPKEKSLVLVKLEPWLCRAYLEAVQREGVSSLDSGS
LSLCLSSSNSLYEDLEHFLEHFLMEVEQAA

The term “SPI1” as used herein refers to Transcription factor PU.1. In one embodiment, SPI1 is of bovine origin. In another embodiment, SPI1 has the GenBank Accession NM_001192133 and the coding/amino acid sequence:

(SEQ ID NO: 8)
MLQACKMEGFPLVPPQPSEDLVPYDTDLYQRQTHEYYPYLSSDGESHSDHY
WDFHPHHVHSEFESFPENHFTELQSVQPPQLQQLYRHMELEQMHVLEPPMA
PPHANLSHQVYLPRMCLPYPSLSPARPSSDEEEGERQSPPLEVSDGEADGL
EPGPGLLHGETGSKKKIRLYQFLLDLLRSGDMKDSIWWVDKDKGTFQFSSK
HKEALAHRWGIQKGNRKKMTYQKMARALRNYGKTGEVKKVKKKLTYQFSGE
VLGRGGLAERRHPPH

The term “FOXO3” as used herein refers to forkhead box O3. In one embodiment, FOXO3 is of bovine origin. In another embodiment, FOXO3 has the GenBank Accession NM_001206083 and the coding/amino acid sequence:

(SEQ ID NO: 9)
MAEAPASPAPISPLEVELDPEFEPQSRPRSCTWPLQRPELQGSPAKPSGEA
AADSMIPEEEDDEDDEDGGGRAGSAMAIGGGGGGPLGSGLLLEDSARLLAP
GGQDPGSGPAPAAGALSGGTQTPLQPQQPLPPPQPGTAGGSGQPRKCSSRR
NAWGNLSYADLITRAIESSPDKRLTLSQIYEWMVRCVPYFKDKGDSNSSAG
WKNSIRHNLSLHSRFMRVQNEGTGKSSWWIINPDGGKSGKAPRRRAVSMDN
SNKYTKSRGRAAKKKAALQTAPESADDSPSQLSKWPGSPTSRSSDELDAWT
DFRSRTNSNASTVSGRLSPILASTELDDVQDDDAPLSPMLYSSSASLSPSV
SKPCTVELPRLTDMAGTMNLNDGLADNLMDDLLDNIALPASQPSPPGGLMQ
RSSSFPYTTKGSGLGSPTSSFSSAVFGPSSLNSLRQSPMQTIQENKPATFS
SMSHYGNQTLQDLLTSDSLSHSDVMMTQSDPLMSQASTAVSAQNSRRNVML
RSDPMMSFAAQPNQGSLVNQNLLHHQHQTQGALGGSRALSNSVSNMGLSDS
SSLGSAKHQQQSPVSQSMQTLSDSVSGSSLYSTSANLPVMGHEKFPSDLDL
DMFNGSLECDMESIIRSELMDADGLDFNFDSLISTQNVVGLNVGSFTGAKQ
ASSQSWVPG

The term “REL” as used herein refers to proto-oncogene c-Rel. In one embodiment, REL is of bovine origin. In another embodiment, REL has the GenBank Accession NM_001192970 and the coding/amino acid sequence:

(SEQ ID NO: 10)
MASGGFNPCIEIIEQPRQRGMRFRYKCEGRSAGSIPGEHSTDNNRTYPSIQ
ILNYYGKGKVRITLVTKNDPYKPHPHDLVGKDCRDGYYEAEFGQERRPLFF
QNLGIRCVKKKEVKDAVISRVRAGINPFNVPEQQLLDIEDCDLNVVRLCFQ
VFLPDEHGNLTTALPPVVSNPIYDNRAPNTAELRICRVNKNCGSVKGGDEI
FLLCDKVQKDDIEVRFVLNDWEAKGVFSQADVHRQVAIVFKTPPYCKAIIE
PVTVKMQLRRPSDQEVSESMDFRYLPDEKDTYGNKAKKQKTTLLFHKLWQD
CGVNFPERPRPSPLGPTGEGRFIKKEPNLFSHGAVLPETSRPVSSQAESYY
SSSASISSTLSHPASAMLPMGTQSSSGWSSVAHPTSRSVNTNSLSSFSTGT
LSSNSQVIPPFLEMSDLNVSNACIYNNTNDIGRMEASSVSPADLYSISDAS
MLPNCPVNMITPSNDSMRETDNPRLVSMNLENPSCNSVLDPRDLRQLHQMS
PSSMSTVTSSSTTAYVAQSEAFEGSDFNCADNSMINEAGPSNSTNANSHGF
GPNSQYSGIGAMQNEQLSDSFAFEFFKVNL

The term “NFAT5” as used herein refers to nuclear factor of activated T-cells 5. In one embodiment, NFATS is of bovine origin. In another embodiment, NFAT5 has the GenBank Accession XM_002694839 and the coding/amino acid sequence:

(SEQ ID NO: 11)
MPSDFISLLSADLDLESPKSLYSRDSLKLHPSQNFHRAGLLEESVYDLLPK
ELQLPPSRETPVASMSQTSGGEAGSPPPAVVAADASSAPSSSSMGGACSSF
TTSSSPTIYSTSVTDSKAMQVESCSSALGVSNRGVSEKQLTSNTVQQHPST
PKRHTVLYISPPPEDLLDNSRMSCQDEGCGLESEQSCSMWMEDSPSNFSNM
STSSYNDNTEVPRKSRKRNPKQRPGVKRRDCEESNMDIFDADSAKAPHYVL
SQLTTDNKGSSKAGNGTLENQKGTGVKKSPMLCGQYPVKSEGKELKIVVQP
ETQHRARYLTEGSRGSVKDRTQQGFPTVKLEGHNEPVVLQVFVGNDSGRVK
PHGFYQACRVTGRNTTXCKEVDIEGTTVIEVGLDPSNNMTLAVDCVGILKL
RNADVEARIGIAGSKKKSTRARLVFRVNITRKDGSTLTLQTPSSPILCTQP
AGVPEILKKSLHSCSVKGEEEVFLIGKNFLKGTKVIFQENVSDENSWKSEA
EIDMELFHQNHLIVKVPPYHDQHITLPVAVGIYVVTNAGRSHDVQPFTYTP
DPAAVALNVNVKKEISSPARPCSFEEAMKAMKTTGCNLDKVNMLPNALITP
LISSTMIKSEDITPMEVTAEKRSPSIFKTTKTVGSTQQTLENLSHIAGNGS
FSSSSSHLTSENEKQQQIQPKAYNPETLTTIQTQDISQPGTFPAVSASSQL
PSNDALLQQATQFQTRETQSREVLQSDGTVVNLSHLTETSQQQQQSPLQEQ
AQTLQQQISSNIFFSPNSVSQQLQNTIQHLQAGSFTGSTASGSNGNVDLVQ
QVLEAQQQLSSVLFSAPDGNENVQEQLSADIFQQVSQIQNSVSPGMFSSTE
PAVHTRPDNLIAGRAESVHPQNENTLSNQQQQQQQQQVMDSSAAMVMEMQQ
SICQAAAQIQSELFPSSASANGNLQQSPVYQQTSHMMSALSANEDMQMQCE
LFSSPPAVSGNETTTTTTQQVATSGTTLFQTSNSGDGEETGAQAKQIQNSV
FQTMVQMQHSGDSQPQVGLFSSTKSMISVQNSGTQQQGNGLFQQGNEMMSL
QSGNFLQQSSHSQAQLFHPQNPIADPQNLSQETQGSIFHSPSPIVHSQTST
ASSEQMQPPMFHSQNTMAVLQGSSVPQDQQSANIFLSQSPMNNLQTNTVAQ
EEQISFFAAQNSISPLQSTSNTEQQAAFQQQAPISHIQTPMLSQEQAQPSQ
QGLFQPQVSLGSLPPNPMPQNQQGTIFQSQHSIVAIQSNSPSQEQQQQQQQ
QQQQQSILFSNQNAMAPMASQKQPPPNMIFNPSQNPVANQEQQNQSIFHQQ
NNMAPMNQEQQPMQFQNQTTVSSLQNPGPAQSESSQTSLFHSSPQIQLVQG
SPSSQEQQVTLFLSPASMSALQTSMNQQDMQQSPLYSPQNNMPGIQGATSS
PQPQATLFHNTTGGTMNQLQNSPGSSQQTSGMFLFGIQNNCSQLLTSGPAT
LPDQLMAISPPGQPQNEGQPPVTTLLSQQMPENSPMASSINTNQNIEKIDL
LVSLQNQGNNLTGSF

In another embodiment, the period of time for exposing the bovine MDMs to a bacterial pathogen in b) is between 12 to 24 hours, optionally about 18 hours. In one embodiment, the test biomarker expression profile further comprises the gene expression level of at least one or more of ATF4, TP63, EGR1, CDKN2A, and RBL1. In another embodiment, the test biomarker expression profile further comprises the gene expression level of at least one or more of MYC, GPNMB, MSR1, DHCR24, and LGMN.

The term “ATF4” as used herein refers to activating transcription factor 4. In one embodiment, ATF4 is of bovine origin. In another embodiment, ATF4 has the GenBank Accession NM_001034342 and the coding/amino acid sequence:

(SEQ ID NO: 12)
MAEMSFLSSEVLGGDFVSPFDQLGLGAEESLGLLDDNLEVAKHFKHHGFSC
DKAKAGSSEWLAVDWLVSDNSKEDAFSGTDWMVEKMDLKEFDFDILFSKDD
LETMPDELLATLDDTCDLFQPLVQETNKEPPQIVNPIGHLPEGLPTIDQGA
PFTFFQPLPPSPGTLSSTPDHSFSLELCSEVVIPEGDSKPDSTTTGFPQCI
KEEDAPSDNDSGICMSPDSSLGSPQDSPSTSRGSPNKSLLSPGALSGSSRP
KPYDPPGEKMVAAKVKGEKLDKKLKKMEQNKTAATRYRQKKRAEQEALTGE
CKELEKKNEALKEKADSLAKEIQYLKDQIEEVRKAREKKRVL

The term “TP63” as used herein refers to tumor protein p63. In one embodiment, TP63 is of bovine origin. In another embodiment, TP63 has the GenBank Accession NM_001191337 and the coding/amino acid sequence:

(SEQ ID NO: 13)
MNFETSRCATLQYCPDPYIQRFVETPAHFSWKESYYRSTMSQSTQTSEFLS
PEVFQHIWDFLEQPICSVQPIDLNFVDEPSENGATNKIEISMDCIRMQDSD
LGDPMWPQYTNLGLLNSMDQQIQNGSSSTSPYNTDHAQNSVTAPSPYAQPS
STFDALSPSPAIPSNTDYPGPHSFDVSFQQSSTAKSATWTYSTELKKLYCQ
IAKTCPIQIKVMTPPPQGAVIRAMPVYKKAEHVTEVVKRCPNHELSREFNE
GQIAPPSHLIRVEGNSHAQYVEDPITGRQSVLVPYEPPQVGTEFTTVLYNF
MCNSSCVGGMNRRPILIIVTLETRDGQVLGRRCFEARICACPGRDRKADED
SIRKQQVSDSTKNGDGTKRPFRQNTHGIQMTSIKKRRSPDDELLYLPVRGR
ETYEMLLKIKESLELMQYLPQHTIETYRQQQQQQHQHLLQKQTSMQSQSSY
GNSSPPLNKMNSMNKLPSVSQLINPQQRNALTPTTIPDGMGANIPMMGTHM
PMAGDMNGLSPTQALPPPLSMPSTSHCTPPPPYPTDCSLVSFLARLGCSSC
LDYFTTQGLTTIYQIEHYSMDDLASLKIPEQFRHAIWKGILDHRQLHDFSS
PPHLLRTPSGASTVSVGSSETRGERVIDAVRFTLRQTISFPPRDEWNDFNF
DMDARRNKQQRIKEEGE

The term “EGR1” as used herein refers to early growth response 1. In one embodiment, EGR1 is of bovine origin. In another embodiment, EGR1 has the GenBank Accession NM_001045875 and the coding/amino acid sequence:

(SEQ ID NO: 14)
MAAAKAEMQLMSPLQISDPFGSFPHSPTMDNYPKLEEMMLSNGAPQFLGAA
GAPEGSSGSSSGSSGGGGGGGGGSSSSNSNSSSAFNPQGEASEQPYEHLTA
ESFPDISLNNEKVLVETSYPSQTTRLPPITYTGRFSLEPAPNSGNTLWPEP
LFSLVSGLVSMTNPPATSSSASSPAASSSASQSPPLSCAVQSNDSSPIYSA
APTFPTPNTDIFPEPQGQAFPGSAGPALQYPPPAYPGAKGGFQVPMIPDYL
FPQQQGDLGLGTPDQKPFQGLESRTQQPSLTPLSTIKAFATQSGSQDLKAL
NSTYQSQLIKPSRMRKYPNRPSKTPPHERPYACPVESCDRRFSRSDELTRH
IRIHTGQKPQCRISMRNFSRSDHLTTHIRTHTGEKPFACDICGRKFARSDE
RKRHTKIHLRQKDKKADKSAASAATSSLPSYPSPVATSYPSPATTSYPSPA
TTSYPSPVPTSYSSPGSSTYPSPVHNGFPSPSVATTYSSVPPAFPTQVSSF
PSSAVTNSFSASTGLSDMTTTFSPRTIEIC

The term “CDKN2A” as used herein refers to cyclin-dependent kinase inhibitor 2A. In one embodiment, CDKN2A is of bovine origin. In another embodiment, CDKN2A has the GenBank Accession XM_010807758 and the coding/amino acid sequence:

(SEQ ID NO: 15)
MVRRFLITVRIRRANGPPRVRIFVVHIARAAGEWAAPSVRAAVALVLMASE
EPAQSAAMHPRPGDDDGQRPRGRAAAAPRRGPQLRGPRHPHPTGARRRPGG
LPGHAGGPAPSWSAAGCARCLGPPARGPGGGAGPPRRRPVPARGCRGHGRR

The term “RBL1” as used herein refers to RB transcriptional corepressor like 1. In one embodiment, RBL1 is of bovine origin. In another embodiment, RBL1 has the GenBank Accession NM_001192602 and the coding/amino acid sequence:

(SEQ ID NO: 16)
MDEDDPHAEGAAVVAAAGEALQALCQELNLDEGSAAEALDDFTAIRGNYSL
EGEVIHWLACSLYVACRKSIIPTVGKGIMEGNCVSLTRILRSAKLSLIQFF
SKMKKWMDMSNLPQEFRERIERLERNFEVSTVIFKKFEPIFLDIFQNPYEE
PPKLPRSRKQRRIPCSVKELFNFCWTLFVYTKGNFRMIGDDLVNSYHLLLC
CLDLIFANAIMCPNRQELLNPSFKGLPSNFQTADFRASEEPPCIIPVLCEL
HDGLLVEAKGIKEHYFKPYISKLFDRKILKGECLLDLCSFTDNSKAVNKEY
EEYVLTVGDFDERIFLGADAEEEIGTPRKFTGDGPLGKLTAQANVECNLQH
HFEKKTSFAPSTPLTGRRYLREKEAVITPVASATQSVSRLQSIVAGLKNAP
SEQLINIFESCMRNPMENIMKIVKGIGETFCQHYTQSTDEQPGSHIDFAVN
RLKLAEILYYKILETVMVQETRRLHGMDMSVLLEQDIFHHSLMACCLEIVL
FAYSSPRTFPWIIEVLNLRPFYFYKVIEVVIRSEEGLSRDMVKHLNSIEEQ
ILESLAWSHDSALWEALQASENRVPTCEEVIFPNNFETGSGGNVQGHLPMM
PMSPLMHPRVKEVRTDSGSLRKDMQPLSPISVHERYSSPTAGSAKRRLFGE
DPPKEILMDRIITEGTKLKIAPSSSITAENISISPGHSLLTMATAIVAGTT
GHKVTIPLHGIANDAGEITLIPISMNTTQESKVESPVSLTAQSLIGASPKQ
THLTKAQEVHPIGISKPKRTGSLALFYRKVYHLASVRLRDLCLKLDVSNEL
RRKIWTCFEFTLVHCPDLMKDRHLDQLLLCAFYIMAKVTKEERTFQEIMKS
YRNQPQANSHVYRSVLLKSIPREVVAYSKNLNGDFEMTDCDLEDATKTPDC
SSGPVKEERGDLIKFYNTIYVGRVKSFALKYDLSNQDHVMEAPPLSPFPHI
KQQPGSPRRISQQHSIYVSPHKNGSGLTPRSALLYKFNGSPSKSLKDINNM
IRQGEQRTKKRAITIDGDAESPAKRLCQENDDVLLKRLQDVVSERANH

The term “MYC” as used herein refers to MYC proto-oncogene, bHLH Transcription Factor. In one embodiment, MYC is of bovine origin. In another embodiment, MYC has the GenBank Accession NM_001046074 and the coding/amino acid sequence:

(SEQ ID NO: 17)
MPLNVSFANKNYDLDYDSVQPYFYCDEEENFYHQQQQSELQPPAPSEDIWK
KFELLPTPPLSPSRRSGLCSPSYVAVASFSPRGDDDGGGGSFSSADQLEMV
TELLGGDMVNQSFICDPDDETLIKNIIIQDCMWSGFSAAAKLVSEKLASYQ
AARKDGGSPSPARGHGGCSTSSLYLQDLSAAASECIDPSVVFPYPLNDSSS
PKPCASPDSTAFSPSSDSLLSSAESSPRASPEPLALHEETPPTTSSDSEEE
QEDEEEIDVVSVEKRQPPAKRSESGSPSAGSHSKPPHSPLVLKRCHVSTHQ
HNYAAPPSTRKDYPAAKRAKLDSGRVLKQISNNRKCASPRSSDTEENDKRR
THNVLERQRRNELKRSFFALRDQIPELENNEKAPKVVILKKATAYILSVQA
EQQKLKSEIDVLQKRREQLKLKLEQIRNSCA

The term “GPNMB” as used herein refers to glycoprotein nmb. In one embodiment, GPNMB is of bovine origin. In another embodiment, GPNMB has the GenBank Accession NM_001038065 and the coding/amino acid sequence:

(SEQ ID NO: 18)
MECLYCFLGFLLLAAGLPLDAAKRFHDVLSNERPSGYMREHNQLSGWSSDE
NDWNEKLYPVWKRGDSRWKSSWKGGRVQAVLTSDSPALVGSTITFAVNLVF
PRCQKEDASGNIVYEKNCRNDTGASPDLYVYNWTAGTEDSDWGNDTSEGHH
NVFPDGKPFPRPWKKNFVYVFHTLGQYFQKLGQCSVTISINTANVSLGPQI
MEVTVYRRHRRAYVPIAKVKDVYVVTDQIPVFVTMSQKNNRNSSDETFLRD
LPITFSVLIHDPSHFLNESAIYYKWNFGDNTGLFVSNNHTLNHTYVLNGTF
SLNLTVQAEVPGPCPLPSPRPPKTTPPLVTAGDSTLELREIPDESCHITRY
GYFKATITIVEGILEVNIIQVTDVPMPRPQPDNSLVDFVVTCHGSIPTEVC
TIISDPSCQITQNPVCDPVAMELGDTCLLTVRRAFSGSGTYCMNLTLGNDA
SLALTSTLVSINSRDPASLLRTANGILVSLGCLAILVTVIAFLMYKKHKEY
KPIENSPGIVIRGKGLNVFLNHAKTLFFPGNQEKDPLLKNQPGIL

The term “MSR1” as used herein refers to macrophage scavenger receptor 1. In one embodiment, MSR1 is of bovine origin. In another embodiment, MSR1 has the GenBank Accession NM_001113240 and the coding/amino acid sequence:

(SEQ ID NO: 19)
MAQWDDFPDQQEDTDSCTESVKFDARSVTALLPPHPKNGPTLQERMKSYKT
ALITLYLIVFVVLVPIIGIVAAQLLKWETKNCTVGSVNADISPSPEGKGNG
SEDEMRFREAVMERMSNMESRIQYLSDNEANLLDAKNFQNFSITTDQRFND
VLFQLNSLLSSIQEHENIIGDISKSLVGLNTTVLDLQFSIETLNGRVQENA
FKQQEEMRKLEERIYNASAEIKSLDEKQVYLEQEIKGEMKLLNNITNDLRL
KDWEHSQTLKNITLLQGPPGPPGEKGDRGPPGQNGIPGFPGLIGTPGLKGD
RGISGLPGVRGFPGPMGKTGKPGLNGQKGQKGEKGSGSMQRQSNTVRLVGG
SGPHEGRVEIFHEGQWGTVCDDRWELRGGLVVCRSLGYKGVQSVHKRAYFG
KGTGPIWLNEVFCFGKESSIEECRIRQWGVRACSHDEDAGVTCTT

The term “DHCR24” as used herein refers to 24-dehydrocholesterol reductase. In one embodiment, DHCR24 is of bovine origin. In another embodiment, DHCR24 has the GenBank Accession NM_001103276 and the coding/amino acid sequence:

(SEQ ID NO: 20)
MEPAVSLAVCALLFLLWVRVKGLEFVLIHQRWVFVCLFLLPLSLIFDIYYY
VRAWVVFKLSSAPRLHEQRVRDIQKQVREWKEQGSKTFMCTGRPGWLTVSL
RVGKYKKTHKNIMINLMDILEVDTKKQIVRVEPLVTMGQVTALLTSIGWTL
PVLPELDDLTVGGLIMGTGIESSSHRYGLFQHICTAYELVLADGSFVRCTP
MENSDLFYAVPWSCGTLGFLVAAEIRIIPAKKYIKLRFEPVRGLEAICDKF
THESQQPENHFVEGLLYSLHEAVIMTGVMTDEAEPSKLNSIGNYYKPWFFK
HVENYLKTNREGLEYIPLRHYYHRHTRSIFWELQDIIPFGNNPIFRYLFGW
MVPPKISLLKLTQGETLRKLYEQHHVVQDMLVPMKCLPQALHTFHNDIHVY
PIWLCPFILPSQPGLVHPKGDEAELYVDIGAYGEPRVKHFEARSCMRQLEK
FVRSVHGFQMLYADCYMDREEFWEMFDGSLYHRLRKQLGCQDAFPEVYDKI
CKAARH

The term “LGMN” as used herein refers to legumain. In one embodiment, LGMN is of bovine origin. In another embodiment, LGMN has the GenBank Accession NM_174101 and the coding/amino acid sequence:

(SEQ ID NO: 21)
MIWEFTVLLSLVLGTGAVPLEDPEDGGKHWVVIVAGSNGWYNYRHQADACH
AYQIVHRNGIPDEQIIVMMYDDIANSEDNPTPGIVINRPNGSDVYQGVLKD
YTGEDVTPKNFLAVLRGDAEAVKGVGSGKVLKSGPRDHVFVYFTDHGATGI
LVFPNEDLHVKDLNETIRYMYEHKMYQKMVFYIEACESGSMMNHLPPDINV
YATTAANPRESSYACYYDEQRSTFLGDWYSVNWMEDSDVEDLTKETLHKQY
QLVKSHTNTSHVMQYGNKSISAMKLMQFQGLKHQASSPISLPAVSRLDLTP
SPEVPLSIMKRKLMSTNDLQESRRLVQKIDRHLEARNIIEKSVRKIVTLVS
GSAAEVDRLLSQRAPLTEHACYQTAVSHFRSHCFNWHNPTYEYALRHLYVL
VNLCENPYPIDRIKLSMNKVCHGYY

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLE 1

Identifying phenotypic variation in functional traits is the primary requirement to identify and understand the genetic mechanisms that shape the trait. As an initial step toward this goal, it was hypothesized that there is considerable variation in the function of bovine Monocyte-Derived Macrophages (MDMs) following in-vitro exposure to bacterial pathogens and that it would be possible to identify individuals which respond stronger than others. Macrophages are a key component of the innate immune system that play a crucial role in the early phase of inflammation (Dunster, 2016; Hamidzadeh et al., 2017). Therefore, the objective of this study was to examine an in-vitro model of phenotyping bovine MDMs, and to evaluate phenotypic and genetic variance. As indicators of MDMs function, phagocytosis and nitric oxide (NO⁻) production were evaluated following exposure of MDMs to two common bacterial pathogens of dairy cows, Escherichia coli (E. coli) and Staphylococcus aureus (S. aureus).

Materials and Methods

Animals

Cows were selected from the research herd at the University of Guelph. This research herd is approximately four times larger than the average commercial dairy herds in Canada. All the cows are registered with Holstein Canada, and health records and treatments are recorded in detail. Hence, samples from this herd provided genetic diversity as well as detailed environmental records for the statistical analysis. The pedigree of the herd was obtained from the Canadian Dairy Network and the relationship between the cows was tested and full-sib animals were removed from the study to maximize host genetic variation of the samples. Sixteen milking cows that were offspring of 12 sires, 15 dams, 9 paternal grandsires and 14 maternal grandsires were selected. These animals were not diagnosed nor treated for any diseases in the lactation period that samples were collected. Samples were collected in groups of four cows per sampling day. For the second part of the study, an additional 58 samples (offspring of 33 sires, 57 dams, 23 paternal grandsires and 37 maternal grandsires) were collected from the same barn with similar criteria to calculate genetic parameters, specifically the heritability of MDMs nitric oxide (NO-) response. All the procedure and handling of the animals were approved by the animal care committee of the University of Guelph.

In-vitro Transformation of Monocyte to Macrophages

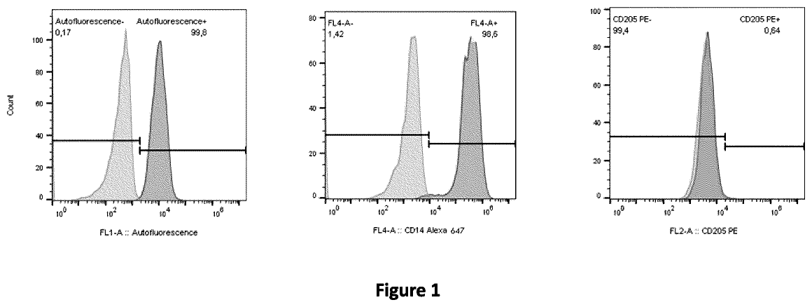

Blood samples were collected from the tail vein in tubes containing EDTA. Blood Mononuclear Cells (BMCs) were purified based on the gradient centrifuge separation method. Concisely, Histopaque-1077 (Sigma-Aldrich, St. Louis, Mo.) was loaded into the Sepmate tubes (STEMCELL Technologies, Vancouver, BC) and whole blood samples were overlaid on the top of Histopaque-1077. After centrifugation for 10 minutes at 1200× g, the layer of cells above the Histopaque-1077 was collected and washed 3 times to obtain the purified BMCs. Purified BMCs were cultured at the concentration of 1×10⁶ cells per square centimetre of the culture flask for 2 hours in Monocyte Attachment Medium (PromoCell, Heidelberg, Germany) at 37° C. Non-adherent cells were removed by washing and the medium was replaced with AIM V® Medium (Thermo Fisher Scientific Inc., Mississauga, ON) supplemented with the reagents in Table 1 containing 5 ng/ml recombinant bovine Granulocyte-macrophage colony-stimulating factor (GM-CSF, Kingfisher Biotech, St. Paul, Minn.; chosen based on maximizing the number of harvested cells at the lowest concentration of GM-CSF in a titration experiment using 2.5, 5 and 10 ng/ml) in the presence of 5% CO₂. After 6 days of incubation, adherent cells were detached from the flask using TrypLE™ Select Enzyme (Thermo Fisher Scientific Inc., Mississauga, ON). Phenotypic characteristics of macrophages (CD14⁺, CD205⁻ and strong auto-fluorescence) were analyzed using flow cytometry to determine the proportion of macrophages among the harvested cells (Njoroge et al., 2001; Fuentes-Duculan et al., 2010; Mitchell et al., 2010). Harvested cells were labelled with RPE conjugated mouse anti-bovine CD205 (Clone: IL-A114), to check the presence of monocyte-derived dendritic cells and ALEXA FLUOR° 647 conjugated mouse anti-human CD14 (Clone: TUK4) to check the presence of myeloid cells, separately. The labelled samples were analyzed using the BD Accuri C6 flow cytometer (Becton Dickinson, Franklin Lakes, N.J.) and the data from the flow cytometer were analyzed by FlowJo V.10 (FlowJo LLC, Ashland, Oreg.). The positive gates were defined based on the fluorescence minus one procedure and the isotype controls were not included based on the absence of any report on the non-specific binding of fluorochrome used (Hulspas et al., 2009). The fluorescence emission of unlabeled harvested cells was compared with the emission of unlabeled fresh BMCs in 533/30 filter excited by the blue laser to test the auto-fluorescence as an indicator for macrophages (FIG. 1).

TABLE 1
Supplementary reagents in the transformation medium

| | Concentration | | Concentration | |
|---|---|---|---|---|
| Reagent | Range | | used in Example | |
| 1. recombinant GM-CSF | 1-10 | μg/L | 5 | μg/L |
| 2. Glycine | 0.01-1 | nM | 0.1 | nM |
| 3. L-Alanine | 0.01-1 | nM | 0.1 | nM |
| 4. L-Asparagine | 0.01-1 | nM | 0.1 | nM |
| 5. L-Aspartic acid | 0.01-1 | nM | 0.1 | nM |
| 6. L-Glutamic Acid | 0.01-1 | nM | 0.1 | nM |
| 7. L-Proline | 0.01-1 | nM | 0.1 | nM |
| 8. L-Serine | 0.01-1 | nM | 0.1 | nM |
| 9. Sodium Pyruvate | 0.01-1 | nM | 0.1 | nM |
| 10. Choline chloride | 0.1-10 | mg/L | 1 | mg/L |
| 11. D-Calcium | 0.1-10 | mg/L | 1 | mg/L |
| pantothenate | | | | |
| 12. Folic Acid | 0.1-10 | mg/L | 1 | mg/L |
| 13. Nicotinamide | 0.1-10 | mg/L | 1 | mg/L |
| 14. Pyridoxal | 0.1-10 | mg/L | 1 | mg/L |
| hydrochloride | | | | |
| 15. Riboflavin | 0.01-1 | mg/L | 0.1 | mg/L |
| 16. Thiamine hydrochloride | 0.1-10 | mg/L | 1 | mg/L |
| 17. i-Inositol | 0.2-20 | mg/L | 2 | mg/L |

Phagocytosis

MDMs from each sample were seeded in 12 wells of an opaque 96-well plate at the concentration of 5×10⁴ cells per well in AIM V® Medium containing 5 ng/ml GM-CSF and incubated overnight. Each sample was assigned to 2 challenge groups and 2 control groups, each group including 3 wells. Challenge groups were exposed to either pHrodo™ Green conjugated E. coli (M01:5) (Thermo Fisher Scientific Inc., Mississauga, ON) or pHrodoTM Green conjugated S. aureus (MOI: 10) (Thermo Fisher Scientific Inc., Mississauga, ON) for four hours. One of the control groups was labelled with NucBlueTM Live ReadyProbesTM Reagent (Thermo Fisher Scientific Inc., Mississauga, ON), 20 minutes before the reading time. The plates were washed 3 times and the fluorescence intensity (FI) was measured by a microplate reader (Biotek Instruments Inc., Winooski, Vt.) as an indication of bacterial uptake. pHrodo™ is fluorescent in an acidic environment and therefore Fl represents the bacterial particles in phagolysosomes. The average of Fl from each sample (3 wells) in the challenge group was corrected by the average of the same sample in the cell-only control group. Then, the corrected Fl for each sample was normalized against the average FI of the control group (12 wells) containing the NucBlueTM to adjust for the cell number in each sample.

Nitric Oxide Production

MDMs from each sample were seeded in 3 wells of a 48-well plate at the density of 2×10⁵ cells per well in AIM V® Medium containing 5 ng/ml GM-CSF and incubated overnight. One well was assigned as the control and the other two wells were assigned as treatment groups and were exposed to inactivated E. coli (MOI: 5, determined by titration (MOI 1, 5, 10, and 50) to induce maximum NO⁻ using minimum MOI, the strain was isolated from a mastitic cow by a microbiologist, Dr. P. Boerlin, Ontario Veterinary College) or S. aureus (MOI: 10, determined by titration (MOI 1, 5, 10, and 50) to induce maximum NO⁻ using minimum MOI) (Thermo Fisher Scientific Inc., Mississauga, ON) for 48 hours. Supernatant from each well was collected and the concentration of nitric oxide (NO⁻) was measured with the Measure-iT™ High-Sensitivity Nitrite Assay Kit (Thermo Fisher Scientific Inc., Mississauga, ON). The concentration of NO⁻ for every sample in the challenge group was subtracted by its replicate in the control group.

Statistical Analysis

On the first data set including samples from 16 cows, two functional responses (NO⁻ and phagocytosis) and 2 treatments (E. coli and S. aureus), the Coefficient of Variation (CV) was calculated based on dividing the standard deviation of the response (log2 transformed corrected fluorescence intensity or the nitrate concentration of culture supernatant 48 hours after challenge) by the average of the response for each treatment, separately. In addition, the correlation between the functional characteristics of MDMs was investigated using Spearman's rank-order correlation. These coefficients and their p-values were calculated in SAS (V 9.4) by employing the PROC CORR procedure.

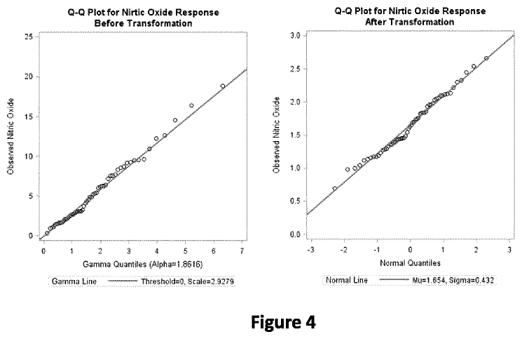

On the second data set including samples from 58 cows, one functional response (NO⁻), and one treatment (E. coli), the PROC UNIVARIATE in SAS (V 9.4) was used to test the distribution of dependent variable (NO⁻ response to E. coli) for normal, Log-normal, Weibull, and Gamma distributions followed by visual inspection of the QQ-plot. The p values for goodness-of-fit tests based on the empirical distribution function, Anderson-Darling, Cramer-von Mises, and Kolmogorov-Smirnov, for Gamma distribution were >0.50, >0.25 and >0.50, respectively. Therefore, the dependent variable was transformed based on the method described by K Krishnamoorthy et al. in 2008 and tested again for normality (FIG. 4) (Krishnamoorthy et al., 2008). The transformed normal data were used in the subsequent statistical analysis. It should be noted that similar procedure was employed on the first data set (n=16). However, no differences were detected on the type of distribution of all four functional responses, probably due to small sample size.

Since all the samples were collected from cows in one farm, only the effect of age in month, days in milking (classes: 1=0-20 days, 2=21-105 days, 3=106-235 days, 4=>235 days), days in pregnancy (classes: 1=non pregnant, 2=1-120 days, 3=121-180 days, 4=181-220 days) (Loker et al., 2009) and sampling group (classes: 1-4 for the first dataset and 1-7 for the second dataset) were tested by

Generalized Linear Model (GLM) procedure in SAS (V 9.4). The statistical model was as follow:

yᵢⱼₖₗₙ=μ+β×aᵢ+gⱼ+mₖ+pₗ+eijkln

where yᵢⱼₗₘ is the phenotypic observation of the n^{th} cow (the cubic roots of nitrite concentration of culture supernatant 48 hours after treatment with E. coli in the second data set (n=58) or raw functional measures on the first data set including phagocytosis and nitric oxide response to E. coli and S. aureus); μ is the overall average of the response. β is the linear coefficient of the fixed regression on age of the cow (aᵢ) (in months); gⱼ is the fixed effect of j^{th} class of sampling group; mₖ is the fixed effect of k^{th} class of days in milking; pi is the fixed effect of I^{th} class of days in pregnancy eᵢⱼₖₗₙ is the random residual effect.

Variance components for NO⁻ production were calculated by the Restricted Maximum Likelihood (REML) method in ASreml package (Ver. 4.1, VSN International) by including the random additive genetic effect of the animal, as in Thompson Crispi et al. (2012b). All available pedigree information was used to calculate the additive genetic relationship matrix fit in the model. Heritability was then calculated dividing the estimated additive genetic variance by the total variance (additive and residual variance).

Results

Functional Characteristics of MDMs

Monocytes are among the first cells that migrate into the infected tissues and give rise to macrophages. MDMs can eliminate the pathogens via phagocytosis and secretion of bactericidal molecules. In this Example, the phagocytic ability of bovine MDMs and the production of NO− were investigated when MDMs were exposed to E. coli and S. aureus. The statistical analysis on the second dataset (n=58) did not show any effect of age (p value=0.37), group of sampling (p value=0.25), days in milking (p value=0.47), and pregnancy (p value=0.73) on the production of NO− in response to E. coli (Model p value=0.52, R-Square=0.21). Similarly, none of these effects were significant on the four functional characteristics of MDMs on the first dataset (n=16).

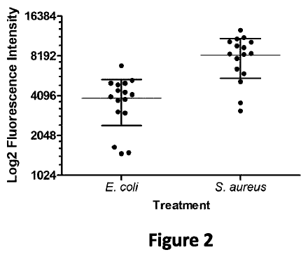

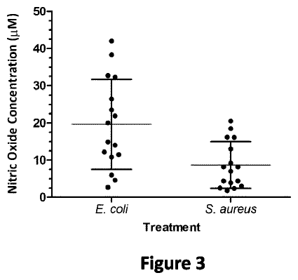

Substantial variation was observed among individuals in phagocytic ability and NO− production of MDMs (FIGS. 2 and 3). The coefficient of variation (CV) ranged from 33% in phagocytic ability to 70% in the production of NO− (inter-assay CV ranged from 6-10% in phagocytosis and 3-9% in NO- production). The maximum concentration of NO− in the supernatant of MDMs was 42 μM in response to E. coli while the highest response to S. aureus was 20.5 μM. It should be noted that one cow had the greatest NO− response to both pathogens. Similarly, MDMs from another cow produced the lowest NO− in response to E. coli and S. aureus, 2.7 and 1.8 μM, respectively. The maximum fluorescence intensity (FI) after phagocytosis of S. aureus belonged to the same individual that produced the highest amount of NO−. The minimum FI after phagocytosis of S. aureus did not belong to the same individual which produced the lowest amount of NO−, but it produced one of the lowest amounts of NO−. To test the repeatability of the results and ensuring the technical assay optimization, NO− response to E. coli was chosen as it showed the highest variation among all measured responses. Blood was collected from four cows initially sampled and the experiment was repeated independently in a double-blind like study when the barn and laboratory personnel was not aware of the results of the first experiment. The correlation coefficient between the two repeats of four cows was 0.97.

Correlation between Functional Characteristics of MDMs

The pairwise rank correlation between the four characteristics of MDMs (phagocytosis and NO⁻ production in response to E. coli and S. aureus) was investigated using Spearman's rank-order test (Table 2). The highest correlation (0.92) was observed between the production of NO⁻ in response to E. coli and S. aureus. The correlation between phagocytosis of E. coli and S. aureus was 0.58. The lowest correlation (0.53) was observed between the phagocytosis of S. aureus and the NO⁻ response to E. coli. These results indicated a strong, significant, and positive correlation among these in-vitro functional characteristics of bovine MDMs. In addition, the heritability of in-vitro NO⁻production in response to E. coli was estimated to be 0.776.

TABLE 2
The results of Spearman's rank correlation test (n = 16) between
phagocytosis and nitric oxide production of E. coli and S aureus.

| | Nitric | | Nitric | |
|---|---|---|---|---|
| | Oxide | | Oxide | |
| | response | Phagocytosis | response | Phagocytosis |
| | to E. coli | of E. coli | to S. aureus | of S. aureus |
| Nitric Oxide | 1 | 0.62 (0.01) | 0.92 (<0.01) | 0.53 (0.03) |
| response to E. coli | | | | |
| Phagocytosis of | | 1 | 0.59 (0.02) | 0.58 (0.02) |
| E. coli | | | | |
| Nitric Oxide | | | 1 | 0.70 (<0.01) |
| response to | | | | |
| S. aureus | | | | |
| Phagocytosis of | | | | 1 |
| S. aureus | | | | |

The Spearman's rank correlation coefficients (rho) between each pair of functional tests are presented in their crossing cell with the p value in the bracket.

Discussion

Macrophages uniquely play dual roles in the immune system. They are professional antigen presenters, as well as effector cells of the immune system (Mills et al., 2015). Macrophages recognize the presence of pathogens via pathogen recognition receptors (PRRs). Following recognition, they destroy the pathogens via phagocytosis and producing respiratory burst-derived microbicidal components, such as reactive oxygen and nitrogen species (Murray and Nathan, 1999; Bogdan et al., 2000; Jordao et al., 2007; Flannagan et al., 2015). Recent advances have shown that monocytes are not necessarily giving rise to tissue-resident macrophages under steady-state in many tissues (Zigmond and Jung, 2013; Gomez Perdiguero et al., 2014; Hoeffel and Ginhoux, 2015; Franken et al., 2016; Ginhoux et al., 2016). However, during inflammation, proinflammatory macrophages that are transformed from monocytes under the influence of GM-CSF play a significant role in controlling infection (Bain and Mowat, 2014; Ginhoux and Guilliams, 2016; Ginhoux et al., 2016). In in-vitro models, blood monocytes can transform to macrophages in the presence of Macrophage Colony-Stimulating Factor (M-CSF) or GM-CSF (Italiani and Boraschi, 2017). Adding M-CSF promotes macrophages towards an anti-inflammatory state, which are also known as M2-like macrophages. These characteristics resemble macrophages under steady-state, but the pitfall in this model is the origin of macrophages, which should not be from blood monocytes. GM-CSF in cattle, mice and humans promotes the transformation of blood monocytes towards proinflammatory macrophages, which is also known as M1-like macrophages. These macrophages resemble the macrophages under inflammation which are of the same origin as monocytes (Soehnlein and Lindbom, 2010). Furthermore, under in-vitro challenge with pathogens in the presence of M-CSF, MDMs receive two signals, pro-inflammatory by Pathogen Associated Molecular Pattern (PAMPs) and anti-inflammatory by M-CSF. This situation is in contrast to what happens in the host during inflammation. During inflammation, specifically at the peak of inflammation, the local tissue milieu contains GM-CSF along with PAMPs from pathogens that stimulate the macrophages in the same direction toward a pro-inflammatory status (Soehnlein and Lindbom, 2010; Becher et al., 2016). Therefore, MDMs in the presence of GM-CSF were chosen as the model. This model seemed to mimic the macrophages in inflamed tissue and exposed to colony stimulating factor that is expressed during inflammation.

Measuring NO⁻ and phagocytosis in in-vitro models has been widely used as an indicator of the bactericidal performance of macrophages, but the results are inconsistent between species. A positive correlation between the concentration of NO⁻ and bactericidal activity has been reported in rats and chickens but not in mice and cattle (Weiss et al., 2002; Sun et al., 2008; Guimaraes et al., 2011; Zhao et al., 2013; Lamont et al., 2014; Akhtar et al., 2016; Qin et al., 2016). Lack of the ability to reliably measure macrophage function, including the production of NO⁻ seems to be the source of inconsistency. Different research groups have frequently observed the complete or partial failure of macrophages in producing NO⁻ in cultures containing fetal bovine serum (FBS). MDMs from humans, cattle, sheep, goats, badgers, and ferrets in a culture containing FBS were found to be unable to produce detectable level of NO⁻ or approximately 20 to 50 fold less than macrophages from chickens (Denis, 1994; Dumarey et al., 1994; Arias et al., 1997; Ohki et al., 1999; Sacco et al., 2006; Zelnickova et al., 2008; Khalifeh et al., 2009; Guimaraes et al., 2011; Azevedo et al., 2016; Bilham et al., 2017; Garcia et al., 2017). FBS contains a considerable amount of immuno-regulatory cytokines and bioactive molecules. In addition, each batch of FBS can contain different components at different concentrations (Zheng et al., 2008; Beninson and Fleshner, 2015). Transforming growth factor-beta (TGF-β) is one of the cytokines that is present in FBS in notable concentrations (Oida and Weiner, 2010). The effect of TGF-β on macrophages in reducing scavenger receptors and suppressing the production of NO⁻ has been shown in previous studies (Becquet et al., 1994; Ohki et al., 1999; Han et al., 2000; Khalifeh et al., 2009; Rey-Giraud et al., 2012; Rath et al., 2014). The similarity and difference between the amino acid sequence of the isoform 2 of TGF-β among cattle (accession number P21214.3), chickens (accession number P30371.1) and humans (accession number P61812.1) can explain the low level of NO⁻ production by human and bovine macrophages in the presence of FBS. The isoform 2 of TGF-β in cattle is 99% identical to its ortholog in humans and only 89% to that of chickens. Subsequently, the effect of TGF-β on human and bovine cells are likely greater compared to chicken. Therefore, a serum-free model was developed to measure the functional performance of MDMs. The high level of NO⁻ produced by MDMs in this culture system and high correlation that was observed between the two repeats of NO⁻ production in response to E. coli showed the robustness of this serum-free culture system.

The considerable phenotypic variation that was observed in the functional characteristics of MDMs among individuals is noteworthy. The statistical analysis revealed no effect of age, days in milking, days in pregnancy and group of sampling on the results of both sample sets (set 1 including 16 cows and set 2 including 58 cows). In addition, the correlation coefficient of NO⁻ production against E. coli in two independent experiments was high (r=0.97). Therefore, it was reasoned that the overall variation that was observed in this trait between individuals is likely due to the genetics of the host. This hypothesis aligned with the high heritability (h2=0.776) that was estimated for the NO⁻ response to E. coli. In addition, the high repeatability and heritability showed that the serum-free culture system and stimulation with GM-CSF is an effective method to evaluate bovine MDMs function in vitro and unmask the genetic effects.

Another notable finding of this study is the rank correlation between the functional characteristics of MDMs. When these functional traits were compared within one pathogen treatment, the rank correlation was significant, moderately high and positive (Table 2). These correlating indices of phagocytosis and NO production, 0.61 for E. coli and 0.70 for S. aureus, suggest that increased bacterial uptake is providing positive signals that in turn upregulate the pathway of NO− production in response to both pathogen treatments. Correlations between phagocytosis and NO− production have been rarely reported and these reports are mainly limited to in-vivo experiments or in-vitro experiments only on MDMs from chickens and mice (de Matos Macchi et al., 2010; Guimaraes et al., 2011).

Additionally, the rank correlation was examined in each functional response (phagocytosis or NO− production) between the two bacterial treatments. In this case, the correlation indices were positive but the difference between the pathogens was large (0.58 for phagocytosis versus 0.92 for NO− production). It should be noted that the comparison beyond rank correlation cannot be justified and it was avoided. The lower FI after phagocytizing E. coli in comparison to S. aureus (FIG. 2) could be due to the intensity of the fluorochrome after labeling the bacterial particle. However, the difference in ranking correlation can be explained by the mechanisms that macrophages employ to recognize the presence of bacteria and initiate phagocytosis versus production of NO−. Gram-positive and negative bacteria display various sets of PAMPs that consist of in-common and Gram-specific PAMPs. The Gram-specific PAMPs are recognized by different sets of PRRs (Mogensen, 2009; Martinez-Florensa et al., 2018). But, NO− is produced through a common pathway following activation of macrophages (Moretti and Blander, 2014; Rath et al., 2014). Therefore, the lower correlations for phagocytosis can best be explained by the utilization of different receptors. In addition, it seems genetic control of the receptors that recognize common PAMPs, such as scavenger receptors, AI and AII, contribute to the moderate phagocytosis correlation that was observed between the two bacterial species (Peiser and Gordon, 2001). The significant and positive correlations between these traits also raise the possibility of using only one of the responses as the indicator of functional characteristics of bovine MDMs as a method to classify cattle based on MDMs function. The strong positive correlation in NO− production (rho=0.92) along with the notable variation that was observed in NO− response to E. coli (CV=70%) suggested it may be suitable to use NO− response to E. coli as a more general indicator of in-vitro bovine MDMs function. Therefore, only NO− response against E. coli was evaluated in the second sample set (n=58 cows). Although the sample size is small in comparison to in-vivo studies on complex traits, when the phenotype is simple, such as gene expression at tissue level in Holstein or NO− response of peritoneal macrophages, approximately 60 samples or less were sufficient to identify expression Quantitative Trait Loci or heritability (Zidek et al., 2000; Higgins et al., 2018). This observation is probably due to the small effective population size of Holstein (Ne ˜115) and the simple genetic nature of the measured traits (Stachowicz et al., 2011; Kemper et al., 2016). In a cellular Genome-Wide Association Study (cGWAS) on human samples, 352 samples were successfully used to investigate the genetic regulation of B lymphocyte response to Salmonella typhi (Alvarez et al., 2017). The Ne of the Utah residents with Northern and Western European ancestry that samples were collected from in the aforementioned cGWAS has been estimated to be 10,437 (PARK, 2011). Therefore, more samples were required to compensate for the genetic variation in human studies.

The analysis of the phenotypic variation revealed that genetics described 77% phenotypic variation of this trait (heritability NO− response to E. coli: 0.776). The heritability of NO− production of peritoneal macrophages in in-vitro system from mice has also been reported to be very high (broad-sense heritability of 0.81) (Zidek et al., 2000). The high heritability does not imply that the environment does not influence MDMs function in other contexts, but rather that the culture method employed here is reliable and consistent enough to reveal the additive genetic variation of this trait. Therefore, this method can be further employed along with omics technology to better understand the genetic control of molecular pathways that shape the response of MDMs.

In conclusion, the serum-free culture method for bovine MDMs developed herein was effective in the evaluation of both phagocytosis and NO⁻ production. The results are similar to in-vivo studies in other species and may provide a feasible approach to measure the activity of inflammatory macrophages in cattle and other large animals in a much less invasive way as compared to broncho-alveolar lavage or tissue biopsies. Without wishing to be bound by theory, the strong positive correlations that were observed between phagocytosis and NO⁻, along with the high heritability of NO⁻ response to E. coli, are likely a reflection of the strong contribution of host genetics on the function of MDMs which has been previously reported in mice and human (Zidek et al., 2000; Motallebipour et al., 2005; Van den Kerkhof et al., 2018). In addition, this model may be used to rank cows based on their MDMs function, to look for disease associations, and to better understand the mechanism(s) that determine the magnitude of these responses in MDMs following bacterial challenge.

### EXAMPLE 2

It is not known if other important characteristics of macrophages such as the profile of cytokine expression, chemotaxis ability, and stimulatory properties of macrophages are different between different functional classes using the NO-index. It has been hypothesized that the inflammatory profile of MDMs differs between classes of macrophages ranked based on NO-production (Lawrence and Natoli, 2011; Murray et al., 2014). In addition, the observed functional variation in the response of MDMs to bacterial treatment is expected to be governed through the differences in the abundance of intracellular signaling components. In the current example, MDMs from cows that were previously classified into high and low responder groups based on the in-vitro NO-response (see Example 1 above) were stimulated with E. coli and the transcriptome of MDMs at two time points were analyzed by RNA-Seq technology. The present inventors identified differentially expressed genes based on in-vitro phenotype, corrected by untreated controls at each time point.

Material and Methods

Monocytes-Derived Macrophages and E. coli Stimulation

Blood samples were collected from the tail vein of 6 Holstein mid-lactating cows from the research herd at the University of Guelph. Based on our previous study (Emam et al., 2019), these samples were divided into high (n=3) and low (n=3) responder groups based on in-vitro NO− response to E. coli. The average of NO− response 48 hours after the challenge was 13.1 μM (Standard Error: 0.66) and 5.3 μM (Standard Error: 0.47) for high and low responder groups, respectively (p-value of T-test between high and low responder groups was <0.002). MDMs were generated in a serum-free culture system by the method previously published (Emam et al., 2019). Briefly, blood Mononuclear Cells (BMCs) were purified based on the gradient centrifuge separation method and cultured for 2 hours in Monocyte Attachment Medium (PromoCell, Heidelberg, Germany) at 37° C. Non-adherent cells were removed and the medium was replaced with AIM V® Medium (Thermo Fisher Scientific Inc., Mississauga, ON) in the presence of the contents of Table 1 and 5% CO2. After six days of incubation, the culture flasks were vigorously washed to remove any dead cells before the harvest. Adherent cells were detached from the flask using TrypLETM Select Enzyme (Thermo Fisher Scientific Inc., Mississauga, ON). Phenotypic characteristics of macrophages (strong auto-fluorescence, CD14+, CD205−) were analyzed using flow cytometry to determine the proportion of macrophages among the harvested cells. MDMs from each sample were seeded in 4 wells in two 24-well plates in AIM V® at the concentration of 0.4×10⁶ cells per well. Each plate was assigned to one time-point. One well of each sample was assigned as the control and the other well was exposed to E. coli (MOI: 5).

RNA Sequencing

At 3 and 18 hours after treatment, total RNA was extracted from all four wells, using TRIzol™ Reagent (Thermo Fisher Scientific Inc., Mississauga, ON) according to the manufacturers protocol. Briefly, 1 ml of Trizol Reagent was used to extract total RNA from 0.4×10⁶ MDMs. The extracted samples were treated with DNase to remove any DNA contamination. The quantity of the purified RNA samples was measured by the RNA High Sensitivity kit in the Qubit Fluorometric Quantification system (Thermo Fisher Scientific Inc., Mississauga, ON) and the qualities were checked by the 2100 Bioanalyzer (Agilent, Santa Clara, Calif.). cDNA libraries were prepared using TruSeq Stranded mRNA Libraries Prep kit (Illumina Inc. San Diego, Calif.), and each sample was labelled with a unique index to make 24 libraries. An equal amount of each library was pooled together and was paired-end sequenced in HiSeq-4000 system (Illumina Inc. San Diego, Calif.) to generate 150 bp reads.

Bioinformatics Analysis

1-Differentially Expressed Genes:

The sequencing reads were filtered for quality and removing the index (universal illumina index) using Trimmomatic in the pair-end mode (Bolger et al, 2014). Nucleotides with quality of less than 30 (in Phred score) at the beginning or the end of the reads were removed. In addition, reads shorter than 100 bp or with quality of less than 20 (in Phred score) in 5 adjacent base pair were removed from the analysis. The quality of the reads was checked by FastQC (v. 0.11.5) before and after trimming (Babraham, 2019). At the next step, clean reads were mapped to the bovine genome (UMD 3.1, Release 94) by using “Spliced Transcripts Alignment to a Reference” (STAR, v. 2.7.0a) (Dobin et al., 2013). The quantity of expression per annotated genes was calculated by using “RNA-Seq by Expectation Maximization” package (RSEM, v. 1.3) (Li et al., 2011). The raw count data were imported by R′ Bioconductor package “tximport” (v 1.10.1) and analyzed by DESeq2 (v. 1.22.2) by employing negative binomial GLM fitting approach (Soneson et al., 2016; Love et al., 2014). Every sample was numbered within a phenotype and consisted of two reads files, treated and control. The model term in DESeq2 was designed to calculate the differential expression in fold change (FC) between the phenotypes in the treated group after accounting for the expression level of the respective control sample. To correct for multiple comparison error, p-values for each gene was adjusted based on the Benjamini and Hochberg method (Benjamini et al.,1995). Genes with the absolute FC of greater than 1.5 and adjusted p-value of less than 0.1 were considered Differentially Expressed (DE).

2-Functional Annotation

The output of DESeq2 analysis was imported to Ingenuity Pathway Analysis (IPA) cloudware (QIAGEN Bioinformatics, Toronto, ON), to identify common Gene Ontology (GO) terms, pathways and networks among the DE genes. The “core analysis” function in IPA was used as per suggested by the manual with following criteria: removing genes with an ambiguated identifier (unmapped), absolute log2 FC of less than 0.58, and q-value of more than 0.1. The Fisher Exact test followed by adjusting p-value based on the Benjamini and Hochberg method was used by the IPA to identify statistically significant enriched GO terms, pathways and their networks.

Results

Differentially Expressed Genes:

At 3 hours after treatment, the average number of reads which passed the quality control was 18.1 and 15.6 million reads per library for the control and treatment group, respectively, with the average size of 289 bp per read. In both groups, more than 96% of the reads were uniquely mapped to the bovine genome. Comparing the gene expression between the two phenotypic groups, 179 genes were identified with the absolute FC of >1.5 and the FDR p-value of less than 0.05. Among these genes, 174 genes had a positive value (were over expressed in the high NO- responder group), 5 genes had a negative value (were more expressed in the low NO- responder group). The average of FC in the top 10 with positive value was 8.55 (average DESeq2 base mean of 926.1) and the average of the 5 DE genes with negative value was 2.39 (average DESeq2 base mean of 568.0).

At 18 hours after treatment, the average number reads which passed the quality control was 21.5 and 21.7 million reads per library for the control and treatment group, respectively, with the average size of 286 bp per read. In both groups, more than 95% of the reads were uniquely mapped to the bovine genome. Comparing the gene expression between the two phenotypic groups, 392 genes were identified with the absolute FC of >1.5 and the FDR p-value of less than 0.05. Among these genes, 326 genes had a positive value (were over expressed in the high NO-responder group), 66 genes had a negative value (were more expressed in the low responder NO-group). The average of FC for the top 10 genes with positive value was 9.32 (average DESeq2 base mean of 1249.7) and the average of the top 10 DE genes with negative value was 3.59 (average DESeq2 base mean of 2435.7). Among differentially expressed genes at 3 and 18 hours, 55 genes were in common.

It is worth emphasizing that the DE genes that were identified in this Example are not only corrected against the untreated control, but the FCs in the expression are calculated based on the differences between two groups of genetically distinct MDMs. These two groups were exposed to the same bacteria with exactly similar MOI in a similar environment. The only difference between these two groups was their genetic architecture as previously shown (Emam et al., 2019).

Functional Annotation

DE genes were annotated and analyzed in IPA. In this analysis, FDR of 0.1 and absolute log2FC of 0.58 (equal to 1.5 FC) were set as the criteria to maximize discovery of the pathways that are associated with different phenotypes. At the first step of the analysis, 8 and 18 canonical pathways were identified with FDR p-value of <0.05 and z score of 2, at 3− and 18-hours post-treatment, respectively. Of note, FC-γ mediated phagocytosis, and 3-phosphoinositide biosynthesis were identified to be positively associated with high responder groups at the first time point (3 hours, FIG. 8). At the 18 hours time point, production of nitric oxide and reactive oxygen species, Th1 pathway, inflammasome pathway and leukocyte extravasation signaling were among the positively associated pathways with MDMs phenotypic groups.

At the next step, all DE genes were screened using IPA to predict the upstream regulators. At both time points, Lipopolysaccharide (LPS) was identified as the most probable stimulator of DE genes with the z-score of 9.00 and p-value of 1e-58 at the second time point. Transcription factors are known to be the master regulators of macrophage functions (Xue et al., 2014; Langlais et al., 2016). After applying a filter to only include transcription factors (0, 9 transcription factors (STAT1, IRF7, SPI1, STAT4, IRF1, HIF1A, FOXO3, REL, and NFAT5) were identified with activation z scores 2, p-value between 1.06e-3 to 1.45e-14 and log2FC >0.58 at the first time point(Table 3). At the second time point, another set of 9 transcription factors (STAT1, IRF1, HIF1A, STAT4, ATF4, TP63, EGR1, CDKN2A, RBL1) were identified with activation z score ≥2, p-value between 3.37e-7 to 1.41e-24 and log2FC ≥0.53 at the second time point (Table 4).

TABLE 3
List of activated/Inhibited transcription factors at 3 hours
after treatment in Monocyte-Derived Macrophages

| Upstream | Expr Log | Predicted | Activation | p-value |
|---|---|---|---|---|
| Regulator | Ratio | Activation State | z-score | of overlap |
| STAT1* | 0.616 | Activated | 4.38 | 1.45E−14 |
| IRF7 | 0.797 | Activated | 3.77 | 4.00E−10 |
| SPI1 | 0.637 | Activated | 3.39 | 1.01E−06 |
| STAT4* | 0.813 | Activated | 3.09 | 8.75E−07 |
| IRF1* | 1.205 | Activated | 2.97 | 3.90E−08 |
| HIF1A* | 0.682 | Activated | 2.27 | 4.45E−05 |
| FOXO3 | 1.603 | Activated | 2.07 | 1.33E−08 |
| REL | 0.937 | Activated | 2.00 | 5.53E−09 |
| NFAT5 | 1.920 | Activated | 2.00 | 1.06E−03 |
| HIC1 | 3.808 | Inhibited | −2.00 | 3.97E−03 |
| IRF4* | 0.650 | Inhibited | −2.25 | 1.00E−06 |

*Transcription factors present at both 3 hours and 18 hours after treatment.

TABLE 4
List of activated/Inhibited transcription factors at 18 hours after
treatment in Monocyte-Derived Macrophages

| | Expr | Predicted | | |
|---|---|---|---|---|
| Upstream | Log | Activation | Activation | p-value of |
| Regulator | Ratio | State | z-score | overlap |
| STAT1* | 0.702 | Activated | 5.448 | 1.41E−24 |
| IRF1* | 1.288 | Activated | 4.552 | 1.16E−16 |
| HIF1A* | 0.529 | Activated | 4.272 | 1.29E−10 |
| STAT4* | 1.015 | Activated | 3.678 | 7.58E−07 |
| ATF4 | 0.571 | Activated | 3.627 | 5.30E−10 |
| TP63 | 1.072 | Activated | 2.816 | 9.41E−04 |
| EGR1 | 0.634 | Activated | 2.606 | 2.70E−05 |
| CDKN2A | 0.574 | Activated | 2.407 | 3.51E−04 |
| RBL1 | −0.848 | Activated | 2.350 | 3.37E−07 |
| E2F1 | −0.905 | Inhibited | −2.005 | 5.56E−10 |
| PRDM1 | 0.812 | Inhibited | −2.415 | 3.48E−14 |
| GATA3 | −1.749 | Inhibited | −2.425 | 2.25E−03 |
| IRF4* | −0.605 | Inhibited | −2.914 | 2.14E−08 |

*Transcription factors present at both 3 hours and 18 hours after treatment.

DE genes were also screened by IPA, to predict cellular process and biological functions that are downstream of DE genes. Among 45 categories of disease and function including 502 different pathways, the top 5 categories based on the proportion of members with z-scores ≥2 were identified as “Free Radical Scavenging” (100%, 3 members), “Cell-To-Cell Signaling and Interaction” (53%, 38 members), “Immune Cell Trafficking” (53%, 32 members), “Cellular Response” (48%, 41 members), “Inflammatory Response” (29%, 51 members), and “Hematological System Development and Function” (23%, 84 members) at 3 hours post-treatment. At 18 hours after treatment, the top 5 categories with most members with z-scores 2 were identified as “Cell-To-Cell Signaling and Interaction” (80%, 60 members), “Immune Cell Trafficking” (78%, 41 members), “Cellular Movement” (68%, 51 members), “Cellular Function and Maintenance” (59%, 39 members), “Hematological System Development and Function” (58%, 96 members). Also, it should be noted that “Inflammatory Response” (48%, 81 members) was the 6th category at 18 hours post-treatment.

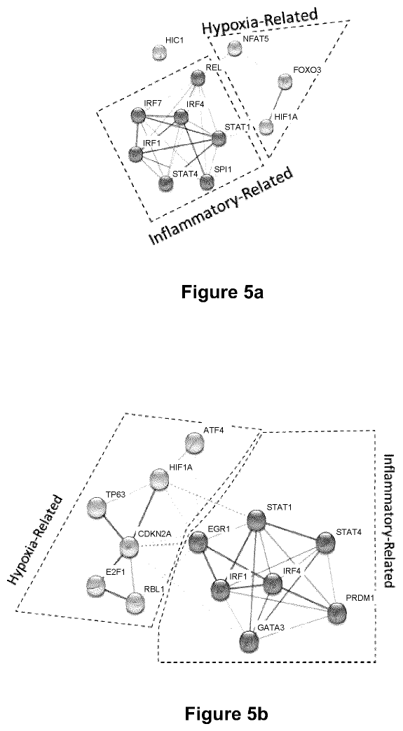

At the last step of the analysis, upstream regulators and downstream impacts along with DE genes were combined in IPA to predict the most probable network of the signaling pathways and biological consequences, simultaneously. The most probable network consisted of 5 transcription regulators, 29 DE genes that are directly linked to them and 6 biological consequences that are directly or indirectly linked to the DE genes and transcription factors (FIG. 5A and FIG. 5B). Downstream Biological impacts that were predicted to be positively associated with the high NO− responder group included: “Antimicrobial Response”, “Antiviral Response”, “Innate Immune Response”, “Activation of Antigen Presenting Cells”, “Activation of Macrophages”. In addition, “Infection of Mammalia” was predicted to be negatively associated with the high responder group (FIG. 5A and FIG. 5B).

Discussion

Reductionist approaches, such as in-vitro models and single-cell analysis, have been instrumental in advancing the understanding of the mechanism that control immune responses by mimicking host-pathogen interactions under experimental challenge designs (Villani et al., 2018). Likewise, Genome-Wide Association Studies have been successful in detecting many Quantitative Trait Loci (QTL) that are associated with complex traits such as disease resistance (Langlais et al., 2017).

However, the combination of these two approaches has recently been explored as an alternative method to investigate the genetic regulation of disease resistance. Expression-QTL (e-QTL) and high-throughput human in-vitro susceptibility testing (Hi-HOST) are just two examples of utilizing the reductionist approach in genetic studies (Goh and Knight, 2017; Miller and Chaudhary, 2016; Langlais et al, 2017; Cookson et al., 2009). As set out herein in Example 1, notable individual variation in NO-production of bovine MDMs. This cellular phenotype was strongly associated with host genetics. The heritability of NO- production was 77%, similar to the heritability of NO-production by peritoneal macrophages in mice (Emam et al., 2019; Zidek et al., 2000). These findings showed that NO production could be considered as a genetic index to classify MDMs. The next step was to investigate if the NO-based classification could represent two functionally distinct groups. Therefore, in this example, the whole transcriptome of MDMs with opposite NO-based phenotypes were compared during the response to E. coli to identify differentially regulated intra-cellular mechanisms and also to investigate if the DE genes could impact functional characteristics of MDMs. Although analyzing the transcriptome reveals an unbiased gene expression, the results are a snapshot in time and cannot represent the dynamic regulation of macrophage functions. This limitation was overcome by employing more than one time point and bioinformatic methods (i.e. in-silico prediction) to expand the snap-shot picture of gene expression in time, retrospectively (upstream regulators) and prospectively (downstream functions).

At the first step of the investigation, more than 170 genes at the 3 hours timepoint and more than 390 genes at the 18 hours time point were found to be significantly associated with NO-production in bovine MDMs. These DE genes were the first indicator of differentially regulated gene expression associated with NO-based classification. Among DE genes, only 55 genes are in common between the two time points. A similar pattern of time-dependent gene expression in bovine MDMs has been reported in response to Mycobacterium avium subspecies paratuberculosis and Mycobacterium bovis (Nalpas et al, 2015; Marino et al. 2017). Although the number of DE genes in the current study seems to be lower than other studies on bovine MDMs (8 to 10 times less than comparable studies), the DE genes and FC reported in the current example are the result of a comparison between two challenged groups, not a challenged group versus a control group (a common design in immunological studies).

It is worth emphasizing that the only difference between the two challenged groups was their genetic background classified based on ability to produce NO−. Therefore, these results indicate that genetics affects gene expression in a time-dependent manner and it should be considered in studies on outbred species, such as human and livestock.

Functional annotation analysis followed by in-silico prediction of the upstream regulator and downstream functions of DE genes revealed some key differences between high and low NO-responder groups. Although some of these results are not surprising, they can indirectly indicate the absence of noise in the data set and the likelihood of accuracy of the obtained results. For instance, at both time points, LPS was found to be the most probable upstream regulator in this experiment by using an unsupervised algorithm by IPA. LPS is one the most abundant Pathogen Associated Molecular Pattern (PAMP) on the surface of Gram-negative bacteria (i.e. E. coli), and it perfectly aligns with the treatment that was used in this study (Kuzmich et al., 2017). Therefore, likely, other upstream regulators were also accurately predicted such as transcription factors. STAT1, STAT4, IRF1 and HIF1A that were predicted in the current study as the upstream regulators associated with NO-Based index (Tables 3 and 4) , are all known key transcription regulators that shape the proinflammatory response of macrophages (Neubert et al., 2019; Ohmori et al., 2001; Kaplan, 2005). The expression of IRF8, IRF1, STAT1 and PU.1 has been shown to be a key regulator of macrophage proinflammatory and antimicrobial response in a mouse model (Langlais et al., 2016). In the current example, IRF1, STAT1 and SPI1 (genes that encode PU.1) were differentially expressed at the 3 hours time point. In addition, the expression of IRF8, IRF1, and STAT1 were differentially expressed at the 18 hours time point. Although it should be noted that FDR p-value of IRF8 and SPI1 was not significant, their unadjusted p-values were less than 0.05. In five species of non-human primates, a conserved regulatory binding site for STAT1, HIF-1, NFAT5 that controls the expression of iNOS has been previously reported (Roodgar et al., 2015). In addition, interactions between HIF-1 and IRF-1, and HIF-1 and STAT1 have also been reported in mice, and these regulate the expression of iNOS and induce apoptosis in cancer cells (Roodgar et al., 2015; Cao et al., 2013). Although, this information supports the association between NO-based phenotypes and these TFs (summarized in FIG. 18) the genetic control in expression of these TFs seems to be less clear. Based on the design of the example, it is possible to infer that the expressions of these TFs are genetically regulated.

Downstream functional impact of DE genes was investigated in two approaches. First, simply by analyzing over-represented pathway terms that are associated with DE genes, reported in canonical pathways in FIGS. 4 and 5. Second, by connecting related biological pathways which are represented as “Diseases and Biological Functions” in Supplementary FIGS. 2 and 3. Among enriched canonical pathways at 3 hours time point, “Fcγ Receptor-mediated Phagocytosis in Macrophages and Monocytes” is notable. Table 2 demonstrates a strong and significant correlation between NO-production and phagocytosis in bovine MDMs. This correlation was not pathogen depended, and it was reported in response to E. coli and S. aureus (Emam et al, 2019). The second most enriched pathway that was positively associated with the high responder group at the 3 hours was VEGF signaling pathway. This pathway regulates angiogenesis and lymphangiogenesis in the host during inflammation. The association between VEGF pathway and macrophages NO− production has been previously reported in mice and plays a vital role in antigen clearance and regulation of inflammation (Corliss et al., 2016; Kimura et al., 2003; Kataru et al., 2009). PI3K signaling was also positively associated with the high responder group. PI3K-dependent pathways have different roles in the cells of the immune system from the regulation of metabolism to down-regulation of inflammation and macrophage polarization (Vergadi et al., 2017; Jellusova et al., 2016). Inhibition of this pathway has resulted in a reduction of proinflammatory cytokine expression in response to LPS in THP-1 derived macrophages (Xie et al., 2014).

At the 18 hours time point, 18 different pathways were found to be positively associated with the high responder group, after applying statistical filters (FDR >0.05 and Z score >2). These pathways are either directly related to proinflammatory responses (i.e. STAT3 (Liu et al., 2018)), iNOS signaling, Inflammasome pathways (Buzzo et al., 2010) and IL-2 Signaling (Qu et al., 2018) or they show inflammatory status in a tissue or an organ (i.e. Neuroinflammatory Signaling pathway). The “Production of Nitric Oxide and Reactive Oxygen Species in Macrophages” pathway had the lowest p-value and “Inflammasome Pathway” was the most enriched pathway (20%) among all pathways. Although it is not surprising to find nitric oxide pathway is enriched in this data set, it indicates the accuracy of the methods that were employed in the current example, from the wet lab (cell culture, stimulation and measuring the NO response) to the bioinformatic analysis (trimming and aligning the reads, quantifying the expression level and thresholds for DE genes). The most enriched pathways at 18 hours timepoint (“Inflammasome Pathway”) is also a known pathway to induce NO− production in macrophages (Buzzo et al., 2010). The epigenetic mechanism of interactions between inflammasome, PARP-1 and iNOS has recently been reported in mice (Buzzo et al., 2017). The inflammasome pathway that is enriched based on DE genes in the current study, its association with the level of NO− production and the stimulator that was used here (E. coli), align nicely with this recent discovery.

Combining of all these pathways resembles a distinct proinflammatory profile between high and low NO− responder groups. “Inflammatory Response”, “Cell-to-Cell Signaling”, “Cellular Movement” and “Immune Cell Trafficking” were predicted to be positively associated with the high responder phenotype at both time points. These predicted functions can constitute a distinct immunological response such as stronger antimicrobial and antiviral responses, a higher level of antigen presentation in the high NO-responder groups which can lead to stronger innate responses and reduced infection (FIG. 5a and FIG. 5b).

Looking at the results through the lens of the macrophage polarization paradigm, macrophages are known to be polarized with distinct characteristics in a continuum of phenotypes from M1 (pro-inflammatory) to M2 (anti-inflammatory), with many stages in between (Lawrence and Natoli, 2011; Murray et al., 2014; Xue to al., 2014). This polarization is classified based on the stimulatory signals that macrophages receive, but this concept has been mainly generated from studies using inbred mice models. Herein, both phenotypes (low and high responders) received the same stimulatory signal (GM-CSF and E. coli under the same environmental condition), but their functional characteristics were distinct. Therefore, genetics adds a third dimension to the linear continuum of the macrophage polarization model. The expression of M1-associated genes such as STAT1 (FC: 1.62, FDR: 0.002), IRF1(FC: 2.44, FDR: 2.83e-11), HIF-1A (FC: 1.44, FDR: 0.046), IL8 (FC: 2.2, FDR: 1.48e-4), CCLS (FC: 5.83, FDR:

0.04), iNOS (FC: 2.26, FDR: 1.89e-4), CD38 (FC: 1.91, FDR: 0.023) and CD14 (FC: 1.60, FDR: 0.005) were found to be significantly more expressed in this high responder group (Lawrence et al., 2011; Murray et al., 2014; da Silva et al., 2017; Amici et al., 2018). Whereas, the expression of M2-associated genes such as MYC (FC: -1.83, FDR: 0.031), GPNMB (FC: −2.0, FDR: 4.34e-4), MSR1 (FC: −1.85, FRD: 0.010), DHCR24 (FC: −1.79, FDR: 0.016) and LGMN (FC: −2.86, FDR: 1.14e-9) are more expressed in low NO- responders (Wang et al., 2018; Gerrick et al., 2018; Labonte et al., 2017; Zhou et al., 2017). It should also be noted that the expression of GATA3 and IRF4, known M2-associated TFs (Murray et al., 2014), were predicted to be inhibited in high responder group (or activated in low responder group) based on DE genes at 18 hours. Based on these results, there is a notable overlap between NO-based classification of bovine MDM and M1/2 macrophage polarization in mice or human. Without wishing to be bound by theory, these results indicate that stimulatory signals are not the sole determinant of macrophages polarization, and the phenotype is shaped in the interaction between genetic and stimulatory signals (also known as gene by environment effects).

In conclusion, the results indicate a distinct proinflammatory profile between MDMs that are classified based on NO-production. It is also predicted that cattle that are classified as high NO-responder will likely mount a stronger innate response and have a lower incidence of infectious disease when NO− is required to help control infection. Moreover, this genetically-depended distinct proinflammatory response might be able to describe the individual differences in the progress of some infectious diseases that are linked to inflammatory responses, such as Johne's disease in cattle or Crohn's disease in human. A notable difference in the progress of these diseases has been reported to be associated with the inflammatory response of the host. Therefore, the NO-based classification is useful for providing a platform to investigate the genetic mechanism(s) that shapes the outcome of host infection.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Abel, L., J. Fellay, D. W. Haas, E. Schurr, G. Srikrishna, M. Urbanowski, N. Chaturvedi, S. Srinivasan, D. H. Johnson, and W. R. Bishai. 2018. Genetics of human susceptibility to active and latent tuberculosis: present knowledge and future perspectives. Lancet Infect. Dis. 18:e64-e75.

Akhtar, R., I. Khan, F. Melzer, H. Neubauer, A. A. Anjum, M. Younus, A. Aslam, and S. Imran. 2016. Differential antibrucella activity of bovine and murine macrophages. Indian J. Exp. Biol. 54:452-456.

Alvarez, M. I., L. C. Glover, P. Luo, L. Wang, E. Theusch, S. H. Oehlers, E. M. Walton, T. T. B. Tram, Y.-L. Kuang, J. I. Rotter, C. M. McClean, N. T. Chinh, M. W. Medina, D. M. Tobin, S. J. Dunstan, and D. C. Ko. 2017. Human genetic variation in VAC14 regulates Salmonella invasion and typhoid fever through modulation of cholesterol. Proc. Natl. Acad. Sci. U. S. A. 201706070.

Amici SA, Young NA, Narvaez-Miranda J, Jablonski KA, Arcos J, Rosas L, Papenfuss TL, Torrelles JB, Jarjour WN, Guerau-de-Arellano M (2018) CD38 Is Robustly Induced in Human Macrophages and Monocytes in Inflammatory Conditions. Front Immunol 9:1593 . doi: 10.3389/fimmu.2018.01593

Arias, M., J. Zabaleta, J. I. Rodriguez, M. Rojas, S.C. Paris, and L.F. Garcia. 1997. Failure to induce nitric oxide production by human monocyte-derived macrophages. Manipulation of biochemical pathways. Allergol. Immunopathol. (Madr). 25:280-8.

Azevedo, M. L. V., N. B. Bonan, G. Dias, F. Brehm, T. M. Steiner, W. M. Souza, A. E. M. fStinghen, F. C. Barreto, S. Elifio-Esposito, R. Pecoits-Filho, and A. N. Moreno-Amaral. 2016. p-Cresyl sulfate affects the oxidative burst, phagocytosis process, and antigen presentation of monocyte-derived macrophages. Toxicol. Lett. 263:1-5.

Andrews S Babraham Bioinformatics-FastQC A Quality Control tool for High Throughput Sequence Data. https://www.bioinformatics.babraham.ac.uk/projects/fastqc/. Accessed 2 May 201

Bain, C. C., and A. M. Mowat. 2014. The monocyte-macrophage axis in the intestine. Cell. Immunol. 291:41-48.

Becher, B., S. Tugues, and M. Greter. 2016. GM-CSF: From Growth Factor to Central Mediator of Tissue Inflammation. Immunity 45:963-973.

Becquet, F., Y. Courtois, and 0. Goureau. 1994. Nitric oxide decreases in vitro phagocytosis of photoreceptor outer segments by bovine retinal pigmented epithelial cells. J. Cell. Physiol. 159:256-262.

Beninson, L. A., and M. Fleshner. 2015. Exosomes in fetal bovine serum dampen primary macrophage IL-1β response to lipopolysaccharide (LPS) challenge. Immunol. Lett. 163:187-192.

Benjamini Y, Hochberg Y (1995) Controlling the False Discovery Rate: A Practical and Powerful Approach to Multiple Testing. J R Stat Soc Ser B 57:289-300. doi: 10.1111/j.2517-6161.1995.tb02031.x

Bilham, K., A. C. Boyd, S. G. Preston, C. D. Buesching, C. Newman, D. W. Macdonald, and A. L. Smith. 2017. Badger macrophages fail to produce nitric oxide, a key anti-mycobacterial effector molecule. Sci. Rep. 7:45470.

Bishop, S. C. 2012. A consideration of resistance and tolerance for ruminant nematode infections. Front. Genet. 3:168.

Bogdan, C., M. Rollinghoff, and A. Diefenbach. 2000. Reactive oxygen and reactive nitrogen intermediates in innate and specific immunity. Curr. Opin. Immunol. 12:64-76.

Bolger A M, Lohse M, Usadel B (2014) Trimmomatic: a flexible trimmer for Illumina sequence data. Bioinformatics 30:2114-20. doi: 10.1093/bioinformatics/btu170

Buzzo CL, Campopiano JC, Massis LM, Lage SL, Cassado AA, Leme-Souza R, Cunha LD, Russo M, Zamboni DS, Amarante-Mendes GP, Bortoluci KR (2010) A novel pathway for inducible nitric-oxide synthase activation through inflammasomes. J Biol Chem 285:32087-95. doi: 10.1074/jbc.M110.124297

Buzzo C de L, Medina T, Branco LM, Lage SL, Ferreira LC de S, Amarante-Mendes GP, Hottiger MO, De Carvalho DD, Bortoluci KR (2017) Epigenetic regulation of nitric oxide synthase 2, inducible (Nos2) by NLRC4 inflammasomes involves PARP1 cleavage. Sci Rep 7:41686. doi: 10.1038/srep41686

Cao Y, Eble JM, Moon E, Yuan H, Weitzel DH, Landon CD, Nien CY-C, Hanna G, Rich JN, Provenzale JM, Dewhirst MW (2013) Tumor cells upregulate normoxic HIF-1α in response to doxorubicin. Cancer Res 73:6230-42. doi: 10.1158/0008-5472.CAN-12-1345

Clapperton, M., A. B. Diack, O. Matika, E. J. Glass, C. D. Gladney, M. A. Mellencamp, A. Hoste, and S. C. Bishop. 2009. Traits associated with innate and adaptive immunity in pigs: heritability and associations with performance under different health status conditions. Genet. Sel. Evol. 41:54.

Cookson W, Liang L, Abecasis G, Moffatt M, Lathrop M (2009) Mapping complex disease traits with global gene expression. Nat Rev Genet 10:184-94. doi: 10.1038/nrg2537

Corliss B A, Azimi MS, Munson JM, Peirce SM, Murfee WL (2016) Macrophages: An Inflammatory Link Between Angiogenesis and Lymphangiogenesis. Microcirculation 23:95-121. doi: 10.1111/micc.12259

da Silva TA, Zorzetto-Fernandes ALV, Cecilio NT, Sardinha-Silva A, Fernandes FF, Roque-Barreira MC (2017) CD14 is critical for TLR2-mediated M1 macrophage activation triggered by N-glycan recognition. Sci Rep 7:7083. doi: 10.1038/s41598-017-07397-0

Denis, M. 1994. Human monocytes/macrophages: NO or no NO?. J. Leukoc. Biol. 55:682-4.

Dobin A, Davis CA, Schlesinger F, Drenkow J, Zaleski C, Jha S, Batut P, Chaisson M, Gingeras TR (2013) STAR: ultrafast universal RNA-seq aligner. Bioinformatics 29:15-21. doi: 10.1093/bioinformatics/bts635

Dumarey, C. H., V. Labrousse, N. Rastogi, B. B. Vargaftig, and M. Bachelet. 1994. Selective Mycobacterium avium-induced production of nitric oxide by human monocyte-derived macrophages. J. Leukoc. Biol. 56:36-40.

Dunster, J. L. 2016. The macrophage and its role in inflammation and tissue repair: mathematical and systems biology approaches. Wiley Interdiscip. Rev. Syst. Biol. Med. 8:87-99.

Emam M, Tabatabaei S, Sargolzaei M, Sharif S, Schenkel F, Mallard B (2019) The Effect of Host Genetics on in vitro Performance of Bovine Monocyte-Derived Macrophages. J Dairy Sci In press:

Flannagan, R. S., B. Heit, and D. E. Heinrichs. 2015. Antimicrobial Mechanisms of Macrophages and the Immune Evasion Strategies of Staphylococcus aureus. Pathog. (Basel, Switzerland) 4:826-68.

Franken, L., M. Schiwon, and C. Kurts. 2016. Macrophages: Sentinels and regulators of the immune system. Cell. Microbiol. 18:475-487.

Fuentes-Duculan, J., M. Suárez-Fariñas, L.C. Zaba, K. E. Nograles, K. C. Pierson, H. Mitsui, C. A. Pensabene, J. Kzhyshkowska, J. G. Krueger, and M. A. Lowes. 2010. A subpopulation of CD163-positive macrophages is classically activated in psoriasis. J. Invest. Dermatol. 130:2412-22.

Garcia, M., Y. Qu, C. M. Scholte, D. O'Connor, W. Rounds, and K. M. Moyes. 2017. Regulatory effect of dietary intake of chromium propionate on the response of monocyte-derived macrophages from Holstein cows in mid lactation. J. Dairy Sci.

Gerrick KY, Gerrick ER, Gupta A, Wheelan SJ, Yegnasubramanian S, Jaffee EM (2018) Transcriptional profiling identifies novel regulators of macrophage polarization. PLoS One 13:e0208602. doi: 10.1371/journal.pone.0208602

Ginhoux, F., and M. Guilliams. 2016. Tissue-Resident Macrophage Ontogeny and Homeostasis. Immunity 44:439-449.

Ginhoux, F., J. L. Schultze, P. J. Murray, J. Ochando, and S. K. Biswas. 2016. New insights into the multidimensional concept of macrophage ontogeny, activation and function. Nat. Immunol. 17:34-40.

Goh C, Knight JC (2017) Enhanced understanding of the host-pathogen interaction in sepsis: new opportunities for omic approaches. Lancet Respir Med 5:212-223. doi: 10.1016/S2213-2600(17)30045-0

Gomez Perdiguero, E., K. Klapproth, C. Schulz, K. Busch, E. Azzoni, L. Crozet, H. Garner, C. Trouillet, M.F. de Bruijn, F. Geissmann, and H.-R. Rodewald. 2014. Tissue-resident macrophages originate from yolk-sac-derived erythro-myeloid progenitors. Nature 518:547-551.

Greives, T. J., N. A. Dochtermann, and E. C. Stewart. 2017. Estimating heritable genetic contributions to innate immune and endocrine phenotypic correlations: A need to explore repeatability. Horm. Behay. 88:106-111.

Guimarães, M. C. C., L. V. C. Guillermo, M. F. de R. Matta, S. G. Soares, and R. A. DaMatta. 2011. Macrophages from chickens selected for high antibody response produced more nitric oxide and have greater phagocytic capacity. Vet. Immunol. Immunopathol. 140:317-322.

Hamidzadeh, K., S. M. Christensen, E. Dalby, P. Chandrasekaran, and D. M. Mosser. 2017. Macrophages and the Recovery from Acute and Chronic Inflammation. Annu. Rev. Physiol. 79:567-592.

Han, J., D. P. Hajjar, J. M. Tauras, J. Feng, A. M. Gotto, and A. C. Nicholson. 2000. Transforming growth factor-betal (TGF-beta1) and TGF-beta2 decrease expression of CD36, the type B scavenger receptor, through mitogen-activated protein kinase phosphorylation of peroxisome proliferator-activated receptor-gamma. J. Biol. Chem. 275:1241-6.

Higgins, M. G., C. Fitzsimons, M. C. McClure, C. McKenna, S. Conroy, D. A. Kenny, M. McGee, S. M. Waters, and D. W. Morris. 2018. GWAS and eQTL analysis identifies a SNP associated with both residual feed intake and GFRA2 expression in beef cattle. Sci. Rep. 8:14301.

Hoeffel, G., and F. Ginhoux. 2015. Ontogeny of Tissue-Resident Macrophages. Front. Immunol. 6:486.

Hulspas, R., M. R. G. O'Gorman, B.L. Wood, J. W. Gratama, and D. R. Sutherland. 2009. Considerations for the control of background fluorescence in clinical flow cytometry. Cytom. Part B Clin. Cytom. 766:355-364.

Italiani, P., and D. Boraschi. 2017. Development and Functional Differentiation of Tissue-Resident Versus Monocyte-Derived Macrophages in Inflammatory Reactions.

Jellusova J, Rickert RC (2016) The PI3K pathway in B cell metabolism. Crit Rev Biochem Mol Biol 51:359-378. doi: 10.1080/10409238.2016.1215288

Jordao, L., C. K. E. Bleck, L. Mayorga, G. Griffiths, and E. Anes. 2007. On the killing of mycobacteria by macrophages. Cell. Microbiol. 0:071106215315001-???

Kaplan MH (2005) STAT4: a critical regulator of inflammation in vivo. Immunol Res 31:231-42. doi: 10.1385/IR:31:3:231

Kataru RP, Jung K, Jang C, Yang H, Schwendener RA, Baik JE, Han SH, Alitalo K, Koh GY (2009) Critical role of CD11b+ macrophages and VEGF in inflammatory lymphangiogenesis, antigen clearance, and inflammation resolution. Blood 113:5650-9. doi: 10.1182/blood-2008-09-176776

Kemper, K. E., M. D. Littlejohn, T. Lopdell, B. J. Hayes, L. E. Bennett, R. P. Williams, X. Q. Xu, P. M. Visscher, M. J. Carrick, and M. E. Goddard. 2016. Leveraging genetically simple traits to identify small-effect variants for complex phenotypes. BMC Genomics 17:858.

Kimura H, Esumi H (2003) Reciprocal regulation between nitric oxide and vascular endothelial growth factor in angiogenesis. Acta Biochim Pol 50:49-59. doi: 035001049

Van den Kerkhof, M., L. Van Bockstal, J. F. Gielis, P. Delputte, P. Cos, L. Maes, G. Caljon, and S. Hendrickx. 2018. Impact of primary mouse macrophage cell types on Leishmania infection and in vitro drug susceptibility. Parasitol. Res. 117:3601-3612.

Vergadi E, leronymaki E, Lyroni K, Vaporidi K, Tsatsanis C (2017) Akt Signaling Pathway in Macrophage Activation and M1/M2 Polarization. J Immunol 198:1006-1014. doi: 10.4049/jimmunol.1601515

Khalifeh, M. S., A. M. Al-Majali, and J.R. Stabel. 2009. Role of nitric oxide production in dairy cows naturally infected with Mycobacterium avium subsp. paratuberculosis. Vet. Immunol. Immunopathol. 131:97-104.

Ko, D. C., T. J. Urban, U. Pozzoli, A. Ferrer-Admetlla, and L. Pattini. 2013. Understanding Human Variation in Infectious Disease Susceptibility through Clinical and Cellular GWAS. PLoS Pathog. 9:e1003424.

Krishnamoorthy, K., T. Mathew, and S. Mukherjee. 2008. Normal-Based Methods for a Gamma Distribution. Technometrics 50:69-78.

Kuzmich NN, Sivak KV, Chubarev VN, Porozov YB, Savateeva-Lyubimova TN, Peri F (2017) TLR4 Signaling Pathway Modulators as Potential Therapeutics in Inflammation and Sepsis. Vaccines 5.: doi: 10.3390/vaccines5040034

Lamont, E. A., A. M. Talaat, P. M. Coussens, J. P. Bannantine, Y. T. Grohn, R. Katani, L. Li, V. Kapur, and S. Sreevatsan. 2014. Screening of Mycobacterium avium subsp. paratuberculosis mutants for attenuation in a bovine monocyte-derived macrophage model. Front. Cell. Infect. Microbiol. 4:87.

Langlais D, Barreiro LB, Gros P (2016) The macrophage IRF8/IRF1 regulome is required for protection against infections and is associated with chronic inflammation. J Exp Med 213:585-603. doi: 10.1084/jem.20151764

Langlais D, Fodil N, Gros P (2017) Genetics of Infectious and Inflammatory Diseases: Overlapping Discoveries from Association and Exome-Sequencing Studies. Annu Rev Immunol 35:1-30. doi: 10.1146/annurev-immunol-051116-052442

Lawrence T, Natoli G (2011) Transcriptional regulation of macrophage polarization: enabling diversity with identity. Nat Rev Immunol 11:750-761. doi: 10.1038/nri3088

Li B, Dewey CN (2011) RSEM: accurate transcript quantification from RNA-Seq data with or without a reference genome. BMC Bioinformatics 12:323. doi: 10.1186/1471-2105-12-323

Liu X, Yin S, Chen Y, Wu Y, Zheng W, Dong H, Bai Y, Qin Y, Li J, Feng S, Zhao P (2018) LPS-induced proinflammatory cytokine expression in human airway epithelial cells and macrophages via NF-KB, STAT3 or AP-1 activation. Mol Med Rep 17:5484-5491. doi: 10.3892/mmr.2018.8542

Loker, S., F. Miglior, J. Bohmanova, J. Jamrozik, and L. R. Schaeffer. 2009. Phenotypic analysis of pregnancy effect on milk, fat, and protein yields of Canadian Ayrshire, Jersey, Brown Swiss, and Guernsey breeds. J. Dairy Sci. 92:1300-1312.

Longley, R., C. Smith, A. Fortin, J. Berghout, B. McMorran, G. Burgio, S. Foote, and P. Gros. 2011. Host resistance to malaria: using mouse models to explore the host response. Mamm. Genome 22:32-42.

Love MI, Huber W, Anders S (2014) Moderated estimation of fold change and dispersion for RNA-seq data with DESeq2. Genome Biot 15:550. doi: 10.1186/s13059-014-0550-8

Mallard, B. A., M. Emam, M. Paibomesai, K. Thompson-Crispi, and L. Wagter-Lesperance. 2015. Genetic selection of cattle for improved immunity and health. Jpn. J. Vet. Res. 63 Suppl 1:S37-44.

Marino R, Capoferri R, Panelli S, Minozzi G, Strozzi F, Trevisi E, Snel GGM, Ajmone-Marsan P, Williams J L (2017) Johne's disease in cattle: an in vitro model to study early response to infection of Mycobacterium avium subsp. paratuberculosis using RNA-seq. Mol Immunol 91:259-271. doi: 10.1016/J.MOLIMM.2017.08.017

Martínez-Florensa, M., C. Català, M. Velasco-de Andres, O. Cañadas, V. Fraile-Ágreda, S. Casadó-Llombart, N. Armiger-Borràs, M. Consuegra-Fernández, C. Casals, and F. Lozano. 2018. Conserved Bacterial-Binding Peptides of the Scavenger-Like Human Lymphocyte Receptor CD6 Protect From Mouse Experimental Sepsis. Front. Immunol. 9:627.

de Matos Macchi, B., J. A. S. Quaresma, A. M. Herculano, M. E. Crespo-Lopez, R. A. DaMatta, and J. L. M. do Nascimento. 2010. Pathogenic action of Plasmodium gallinaceum in chickens: Brain histology and nitric oxide production by blood monocyte-derived macrophages. Vet. Parasitol. 172:16-22.

McManus, C., T. do P. Paim, C. B. de Melo, B.S.A.F. Brasil, and S. R. Paiva. 2014. Selection methods for resistance to and tolerance of helminths in livestock. Parasite 21:56.

Miller S, Chaudhary A (2016) A Cellular GWAS Approach to Define Human Variation in Cellular Pathways Important to Inflammation. Pathogens 5:39. doi: 10.3390/pathogens5020039

Mills, C. D., L. L. Lenz, and K. Ley. 2015. Macrophages at the fork in the road to health or disease. Front. Immunol. 6:59.

Min-Oo, G., M. M. Stevenson, A. Fortin, and P. Gros. 2007. Genetic control of host-pathogen interactions in mice. Novartis Found. Symp. 281:156-65; discussion 165-8, 208-9.

Minozzi, G., J. L. Williams, A. Stella, F. Strozzi, M. Luini, M. L. Settles, J. F. Taylor, R. H. Whitlock, R. Zanella, and H. L. Neibergs. 2012. Meta-Analysis of Two Genome-Wide Association Studies of Bovine Paratuberculosis. PLoS One 7:e32578.

Mitchell, A. J., L. C. Pradel, L. Chasson, N. Van Rooijen, G. E. Grau, N. H. Hunt, and G. Chimini. 2010. Technical advance: autofluorescence as a tool for myeloid cell analysis. J. Leukoc. Biol. 88:597-603.

Mogensen, T. H. 2009. Pathogen recognition and inflammatory signaling in innate immune defenses. Clin. Microbiol. Rev. 22:240-73, Table of Contents.

Moretti, J., and J. M. Blander. 2014. Insights into phagocytosis-coupled activation of pattern recognition receptors and inflammasomes. Curr. Opin. Immunol. 26:100-110.

Motallebipour, M., A. Rada-Iglesias, M. Jansson, and C. Wadelius. 2005. The promoter of inducible nitric oxide synthase implicated in glaucoma based on genetic analysis and nuclear factor binding. Mol. Vis. 11:950-7.

Murray, H. W., and C. F. Nathan. 1999. Macrophage microbicidal mechanisms in vivo: reactive nitrogen versus oxygen intermediates in the killing of intracellular visceral Leishmania donovani. J. Exp. Med. 189:741-6.

Murray PJ, Allen JE, Biswas SK, et al (2014) Macrophage activation and polarization: nomenclature and experimental guidelines. Immunity 41:14-20. doi: 10.1016/j.immuni.2014.06.008

Nalpas NC, Magee DA, Conlon KM, Browne JA, Healy C, McLoughlin KE, Rue-Albrecht K, McGettigan PA, Killick KE, Gormley E, Gordon S V., MacHugh DE (2015) RNA sequencing provides exquisite insight into the manipulation of the alveolar macrophage by tubercle bacilli. Sci Rep 5:13629. doi: 10.1038/srep13629

Neubert P, Weichselbaum A, Reitinger C, et al (2019) HIF1A and NFAT5 coordinate Na+-boosted antibacterial defense via enhanced autophagy and autolysosomal targeting. Autophagy 1-18. doi: 10.1080/15548627.2019.1596483

Njoroge, J. M., L. B. Mitchell, M. Centola, D. Kastner, M. Raffeld, and J. L. Miller. 2001. Characterization of viable autofluorescent macrophages among cultured peripheral blood mononuclear cells. Cytometry 44:38-44.

Ohki, K., F. Amano, S. Yamamoto, and O. Kohashi. 1999. Suppressive effects of serum on the LPS-induced production of nitric oxide and TNF-alpha by a macrophage-like cell line, WEHI-3, are dependent on the structure of polysaccharide chains in LPS. Immunol. Cell Biol. 77:143-152.

Ohmori Y, Hamilton TA (2001) Requirement for STAT1 in LPS-induced gene expression in macrophages. J Leukoc Biol 69:598-604

Oida, T., and H. L. Weiner. 2010. Depletion of TGF-β from fetal bovine serum. J. Immunol. Methods 362:195-198.

PARK, L. 2011. Effective population size of current human population. Genet. Res. (Camb). 93:105-114.

Peiser, L., and S. Gordon. 2001. The function of scavenger receptorsexpressed by macrophages and their rolein the regulation of inflammation. Microbes Infect. 3:149-159.

Qin, Y., Q. Wang, Y. Zhou, Y. Duan, and Q. Gao. 2016. Inhibition of IFN-γ-Induced Nitric Oxide Dependent Antimycobacterial Activity by miR-155 and C/EBPβ. Int. J. Mol. Sci. 17:535.

Qu X, Tang Y, Hua S (2018) Immunological Approaches Towards Cancer and Inflammation: A Cross Talk. Front Immunol 9: doi: 10.3389/fimmu.2018.00563

Rath, M., I. Muller, P. Kropf, E.I. Closs, and M. Munder. 2014. Metabolism via Arginase or Nitric Oxide Synthase: Two Competing Arginine Pathways in Macrophages. Front. Immunol. 5:532.

Rey-Giraud, F., M. Hafner, and C. H. Ries. 2012. In vitro generation of monocyte-derived macrophages under serum-free conditions improves their tumor promoting functions. PLoS One 7:e42656.

Ring, S. C., D. A. Graham, R. G. Sayers, N. Byrne, M. M. Kelleher, M. L. Doherty, and D. P. Berry. 2018. Genetic variability in the humoral immune response to bovine herpesvirus-1 infection in dairy cattle and genetic correlations with performance traits. J. Dairy Sci. 101:6190-6204.

Roodgar M, Ross CT, Kenyon NJ, Marcelino G, Smith DG (2015) Inducible nitric oxide synthase (iNOS) regulatory region variation in non-human primates. Infect Genet Evol 31:236-44. doi: 10.1016/j.meegid.2015.01.015

Sacco, R. E., W. R. Waters, K. M. Rudolph, and M. L. Drew. 2006. Comparative nitric oxide production by LPS-stimulated monocyte-derived macrophages from Ovis canadensis and Ovis aries. Comp. Immunol. Microbiol. Infect. Dis. 29:1-11.

Soehnlein, O. and L. Lindbom. 2010. Phagocyte partnership during the onset and resolution of inflammation. Nat. Rev. Immunol. 10:427-39.

Soneson C, Love MI, Robinson MD (2016) Differential analyses for RNA-seq: transcript-level estimates improve gene-level inferences. F1000Research 4:1521. doi: 10.12688/f1000research.7563.2

Stachowicz, K., M. Sargolzaei, F. Miglior, and F. S. Schenkel. 2011. Rates of inbreeding and genetic diversity in Canadian Holstein and Jersey cattle. J. Dairy Sci. 94:5160-5175.

Sun, S. F., Q. Z. Pan, X. Hui, B. L. Zhang, H. M. Wu, H. Li, W. Xu, Q. Zhang, J. Y. Li, X. M. Deng, J. W. Chen, Z. X. Lian, and N. Li. 2008. Stronger In Vitro Phagocytosis by Monocytes-Macrophages Is Indicative of Greater Pathogen Clearance and Antibody Levels In Vivo. Poult. Sci. 87:1725-1733.

Thompson-Crispi, K. A., B. Hine, M. Quinton, F. Miglior, and B. A. Mallard. 2012a. Short communication: Association of disease incidence and adaptive immune response in Holstein dairy cows. J. Dairy Sci. 95:3888-93.

Thompson-Crispi, K. A., M. Sargolzaei, R. Ventura, M. Abo-Ismail, F. Miglior, F. Schenkel, and B. A. Mallard. 2014. A genome-wide association study of immune response traits in Canadian Holstein cattle. BMC Genomics 15:559.

Thompson-Crispi, K. A., A. Sewalem, F. Miglior, and B. A. Mallard. 2012b. Genetic parameters of adaptive immune response traits in Canadian Holsteins. J. Dairy Sci. 95:401-9.

Villani A-C, Sarkizova S, Hacohen N (2018) Systems Immunology: Learning the Rules of the Immune System. Annu Rev Immunol 36:813-842. doi: 10.1146/annurev-immunol-042617-053035

Wang D, Xiong M, Chen C, Du L, Liu Z, Shi Y, Zhang M, Gong J, Song X, Xiang R, Liu E, Tan X (2018) Legumain, an asparaginyl endopeptidase, mediates the effect of M2 macrophages on attenuating renal interstitial fibrosis in obstructive nephropathy. Kidney Int 94:91-101. doi: 10.1016/j.kint.2017.12.025

Wang, L., K. J. Pittman, J. R. Barker, R. E. Salinas, I. B. Stanaway, G. D. Williams, R. J. Carroll, T. Balmat, A. Ingham, A. M. Gopalakrishnan, K. D. Gibbs, A. L. Antonia, J. Heitman, S. C. Lee, G. P. Jarvik, J. C. Denny, S. M. Horner, M. R. DeLong, R. H. Valdivia, D. R. Crosslin, D. C. Ko, and D. C. Ko. 2018. An Atlas of Genetic Variation Linking Pathogen-Induced Cellular Traits to Human Disease. Cell Host Microbe 24:308-323.e6.

Weiss, D. J., O. A. Evanson, A. Moritz, M. Q. Deng, and M.S. Abrahamsen. 2002. Differential responses of bovine macrophages to Mycobacterium avium subsp. paratuberculosis and Mycobacterium avium subsp. avium. Infect. Immun. 70:5556-61.

Xie S, Chen M, Yan B, He X, Chen X, Li D (2014) Identification of a role for the PI3K/AKT/mTOR signaling pathway in innate immune cells. PLoS One 9:e94496. doi: 10.1371/journal.pone.0094496

Xue J, Schmidt S V., Sander J, et al (2014) Transcriptome-Based Network Analysis Reveals a Spectrum Model of Human Macrophage Activation. Immunity 40:274-288. doi: 10.1016/j.immuni.2014.01.006

Zelnickova, P., J. Matiasovic, B. Pavlova, H. Kudlackova, F. Kovaru, and M. Faldyna. 2008. Quantitative nitric oxide production by rat, bovine and porcine macrophages. Nitric Oxide 19:36-41.

Zerbino, D. R., P. Achuthan, W. Akanni, M. R. Amode, D. Barrell, J. Bhai, K. Billis, C. Cummins, A. Gall, C. G. Giron, L. Gil, L. Gordon, L. Haggerty, E. Haskell, T. Hourlier, O. G. Izuogu, S. H. Janacek, T. Juettemann, J. K. To, M. R. Laird, I. Lavidas, Z. Liu, J. E. Loveland, T. Maurel, W. McLaren, B. Moore, J. Mudge, D. N. Murphy, V. Newman, M. Nuhn, D. Ogeh, C. K. Ong, A. Parker, M. Patricio, H. S. Riat, H. Schuilenburg, D. Sheppard, H. Sparrow, K. Taylor, A. Thormann, A. Vullo, B. Waits, A. Zadissa, A. Frankish, S. E. Hunt, M. Kostadima, N. Langridge, F. J. Martin, M. Muffato, E. Perry, M. Ruffier, D. M. Staines, S. J. Trevanion, B. L. Aken, F. Cunningham, A. Yates, and P. Flicek. 2018. Ensembl 2018. Nucleic Acids Res. 46: D754-D761.

Zhao, Z.-J., J. Zhang, J. Wei, Z. Li, T. Wang, S.-Q. Yi, J.-L. Shen, T.-B. Yang, G. Hide, and Z.-R. Lun. 2013. Lower expression of inducible nitric oxide synthase and higher expression of arginase in rat alveolar macrophages are linked to their susceptibility to Toxoplasma gondii infection. PLoS One 8:e63650.

Zheng, X., H. Baker, W. S. Hancock, F. Fawaz, M. McCaman, and E. Pungor. 2008. Proteomic Analysis for the Assessment of Different Lots of Fetal Bovine Serum as a Raw Material for Cell Culture. Part IV. Application of Proteomics to the Manufacture of Biological Drugs. Biotechnol. Prog. 22:1294-1300.

Zidek, Z., D. Franková, and M. Boubelik. 2000. Genetic variation in in-vitro cytokine-induced production of nitric oxide by murine peritoneal macrophages. Pharmacogenetics 10:493-501.

Zigmond, E., and S. Jung. 2013. Intestinal macrophages: Well educated exceptions from the rule. Trends Immunol. 34:162-168.

1. An in vitro method of generating bovine monocyte-derived macrophages from monocytes comprising
a) culturing bovine monocytes in serum-free media supplemented with granulocyte-macrophage stimulating factor (GM-CSF) to generate the bovine monocyte-derived macrophages (MDMs); and
b) harvesting the bovine MDMs.

2. The in vitro method of claim 1, wherein the GM-CSF is from 1-10 ng/mL, optionally 5 ng/mL.

3. (canceled)

4. (canceled)

5. The in vitro method of claim 1, wherein the serum-free media further comprises 0.01-1 mM glycine, 0.01-1 mM L-alanine, 0.01-1 mM L-asparagine, 0.01-1 mM L-aspartic acid, 0.01-1 mM L-glutamic acid, 0.01-1 mM L-proline, 0.01-1 mM L-serine, 0.01-1 mM sodium pyruvate, 0.1-10 mg/L choline chloride, 0.1-10 mg/L D-calcium pantothenate, 0.1-10 mg/L folic acid, 0.1-10 mg/L nicotinamide, 0.1-10 mg/L pyridoxal hydrochloride, 0.01-1 mg/L riboflavin, 0.1-10 mg/L thiamine hydrochloride and 0.2-20 mg/L i-inositol; or wherein the serum-free media further comprises 0.1 mM glycine, 0.1 mM L-alanine, 0.1 mM L-asparagine, 0.1 mM L-aspartic acid, 0.1 mM L-glutamic acid, 0.1 mM L-proline, 0.1 mM L-serine, 0.1 mM sodium pyruvate, 1 mg/L choline chloride, 1 mg/L D-calcium pantothenate, 1 mg/L folic acid, 1 mg/L nicotinamide, 1 mg/L pyridoxal hydrochloride, 0.1 mg/L riboflavin, 1 mg/L thiamine hydrochloride and 2 mg/L i-inositol.

6. (canceled)

7. The in vitro method of claim 1, wherein culturing in a) comprises culturing the cells for 4-8 days, optionally 6 days.

8. (canceled)

9. (canceled)

10. A method of measuring innate immune response potential of a bovine animal comprising
a) generating bovine MDMs by the method of claim 1;
b) exposing the bovine MDMs to a bacterial pathogen to induce respiratory burst;
c) collecting supernatant of the culture; and
d) measuring molecules that are produced by respiratory burst, such as nitric oxide or a reactive oxygen species, in the supernatant as a measure of innate immune response potential.

11. (canceled)

12. The method of claim 10, wherein b) comprises exposing the bovine MDMs to the bacterial pathogen for 18 to 72 hours, optionally for about 48 hours.

13. (canceled)

14. The method of claim 10, wherein the bacterial pathogen is a live attenuated or inactivated bacteria, such as a Gram-negative or Gram-positive bacteria, optionally wherein the Gram-negative bacteria is E. coli, Klebsiella spp, Serratia spp or Enterobaceter spp. or the Gram-positive bacteria is Staphylococcus spp or Streptococcus spp.

15. (canceled)

16. (canceled)

17. The method of claim 14, wherein the bacteria is inactivated E. coli and the bovine MDMs are exposed to the inactivated E. coli at a multiplicity of infection of 1-50, optionally 5; or wherein the bacteria is inactivated S. aureus and the bovine MDMs are exposed to the inactivated S. aureus at a multiplicity of infection of 1-50, optionally 10.

18. (canceled)

19. (canceled)

20. (canceled)

21. A method of measuring innate immune response potential of a bovine animal comprising
a) generating bovine MDMs by the method of claim 1;
b) exposing the bovine MDMs to fluorescently-labelled bacteria; and
c) measuring bacterial uptake of the MDMs as a measure of innate immune response potential.

22. The method of claim 21, wherein the bacteria is a Gram-negative bacteria, such as E. coli, Klebsiella spp, Serratia spp or Enterobaceter spp, or a Gram-positive bacteria, such as Staphylococcus spp or Streptococcus spp.

23. (canceled)

24. (canceled)

25. (canceled)

26. (canceled)

27. A method of selecting bovine animals for breeding comprising
a) measuring innate immune response potential of a bovine animal by the method of claim 10; and
b) selecting the bovine animal for breeding if the innate immune response potential is high or not selecting a bovine animal for breeding if the innate immune response is low.

28. The method of claim 27, wherein NO production is measured as the measure of respiratory burst and wherein:
a) the innate immune response is high if the NO production is greater than 11 μM when 0.4±0.05e6 cells per 1 cm of culture area in 1 mL of culture medium are used and/or wherein high innate immune response potential is determined as an increase compared to a control; or
b) the innate immune response is low if the NO production is less than 6 uM when 0.4±0.05e6 cells per 1 cm of culture area in 1 mL of culture medium are used and/or wherein low innate immune response potential is determined as a descrease compared to a control.

29. (canceled)

30. (canceled)

31. A method of screening for a gene expression profile for detecting optimal suboptimal innate immune response potential of a bovine animal comprising
a) measuring the gene expression of a blood sample from a bovine animal that has been identified as having high innate immune response potential by the method of claim 10 compared to a control;
b) measuring the gene expression of a blood sample from a bovine animal that has been identified as having low innate immune response potential by the method of claim 10 compared to a control;
c) identifying genes that are differentially expressed between a) and b) to determine the gene expression profile for detecting optimal or suboptimal innate immune response potential of a bovine animal.

32. A method of screening for SNPs for detecting optimal innate immune response potential of a bovine animal comprising
a) determining a SNP profile of a tissue sample from a bovine animal that has been identified as having high innate immune response potential by the method of claim 10 compared to a control;
b) determining a SNP profile of a tissue sample from a bovine animal that has been identified as having low innate immune response potential by the method of claim 10 compared to a control;
c) identifying SNPs that are differentially found in a) and not b) to determine the SNPs for detecting optimal innate immune response of a bovine animal; and
d) identifying SNPS that are differentially found in b) but not a) for detecting suboptimal innate immune response of a bovine animal.

33. The method of claim 31, wherein NO production is measured as the measure of respiratory burst and wherein the innate immune response is high if the NO production is greater than 11 μM when 0.4±0.05e6 cells per 1 cm of culture area in 1 mL of culture medium are used and/or wherein the innate immune response is low if the NO production is less than 6 μM when 0.4±0.05e6 cells per 1 cm of culture area in 1 mL of culture medium are used.

34. The method of claim 32, wherein NO production is measured as the measure of respiratory burst and wherein the innate immune response is high if the NO production is greater than 11 μM when 0.4±0.05e6 cells per 1 cm of culture area in 1 mL of culture medium are used and/or wherein the innate immune response is low if the NO production is less than 6 μM when 0.4±0.05e6 cells per 1 cm of culture area in 1 mL of culture medium are used.

35. (canceled)

36. A method of measuring innate immune response potential of a bovine animal comprising:
a) generating bovine MDMs by the method of claim 1;
b) exposing a test sample of the bovine MDMs to a bacterial pathogen for a period of time;
c) determining a test biomarker expression profile from the test sample, the test biomarker expression profile comprising the level of gene expression of at least one of STAT1, STAT4, iNOS, IRF1, IRF4 and HIFIA; and
d) determining the level of similarity of the test biomarker expression profile to one or more control profiles, wherein
i) a high level of similarity of the test biomarker expression profile to a high-innate control profile or a low level of similarity to a low-innate control profile indicates an increased likelihood of high innate immune response potential of the bovine animal; or
ii) a high level of similarity of the test biomarker expression profile to a low-innate control profile or a low level of similarity to a high innate control profile indicates an increased likelihood of low innate immune response potential of the bovine animal.

37. The method of claim 36, wherein the period of time for exposing the bovine MDMs to a bacterial pathogen in b) is about 3 hours and wherein the test biomarker expression profile further comprises the gene expression level of at least one or more of IRF7, SPI1, FOXO3, REL, and NFAT5.

38. (canceled)

39. The method of claim 36, wherein the period of time for exposing the bovine MDMs to a bacterial pathogen in b) is about 18 hours and wherein the test biomarker expression profile further comprises the gene expression level of at least one or more of ATF4, TP63, EGR1, CDKN2A, and RBL1 and/or wherein the test biomarker expression profile further comprises the gene expression level of at least one or more of MYC, GPNMB, MSR1, DHCR24, and LGMN.

40. (canceled)

41. (canceled)

42. (canceled)

43. The method of claim 36, wherein the bacterial pathogen is a live attenuated or inactivated bacteria, optionally wherein the live attenuated or inactivated bacteria is a Gram-negative bacteria, such as E. coli, Klebsiella spp., Serratia spp., or Enterobaceter spp. or a Gram-positive bacteria, such as Staphylococcus spp. or Streptococcus spp.

44. (canceled)

45. (canceled)

46. (canceled)

47. (canceled)

48. (canceled)

49. (canceled)

50. (canceled)
